(19) United States
(12) Patent Application Publication
(10) Pub. No.: US 2026/0224574 A1
(43) Pub. Date: Aug. 6, 2026

(54) ANTIFUNGAL COMPOSITIONS AND METHODS OF USE THEREOF

(71) Applicant: ARIZONA BOARD OF REGENTS ON BEHALF OF THE UNIVERSITY OF ARIZONA, Tucson, AZ (US)

(72) Inventors: Hongmin Li, Tucson, AZ (US); Anil Tharappel, Tucson, AZ (US); Zhong Li, Tucson, AZ (US)

(21) Appl. No.: 19/156,857

(22) PCT Filed: Feb. 15, 2024
(86) PCT No.: PCT/US2024/016030

§ 371 Date: Aug. 14, 2025

Related U.S. Application Data
(60) Provisional application No. 63/485,396, filed on Feb. 16, 2023.

(51) Int. Cl. A61K 31/519; A61K 31/085; A61K 31/404; A61K 31/4168; A61K 31/4174; A61K 31/427; A61K 31/433; A61K 31/438; A61K 31/506; A61K 31/517; A61P 31/10

(52) U.S. Cl. CPC A61K 31/519; A61K 31/085; A61K 31/404; A61K 31/4168; A61K 31/4174; A61K 31/427; A61K 31/433; A61K 31/438; A61K 31/506; A61K 31/517; A61P 31/10

(57) ABSTRACT

Fungal infections, although not very contagious, affect 300 million people worldwide, with immunocompromised individuals most affected. Candidiasis, cryptococcosis, aspergillosis, and pneumocystosis are major fungal infections. Cryptococcal meningitis is caused by opportunistic fungal pathogens Cryptococcus neoformans (Cne) and Cryptococcus gattii (Cga). Approximately 223,100 human patients are affected by cryptococcosis globally, with 180,000 deaths annually. Cne and Cga initially affect the lungs and, in severe conditions, the brain, leading to meningitis. The present invention screened 98,441 compounds and found 91 compounds with novel anti-Cryptococcus neoformans activity with IC₅₀ less than 10 uM. These compounds represent novel antifungals with the potential to treat cryptococcosis. Thus, compositions and methods for treating a fungal infection (e.g., cryptococcosis) are described herein.

### CROSS-REFERENCE TO RELATED APPLICATIONS

This application claims benefit of U.S. Provisional Application No. 63/485,396 filed Feb. 16, 2023, the specification of which is incorporated herein in their entirety by reference.

### STATEMENT REGARDING FEDERALLY SPONSORED RESEARCH OR DEVELOPMENT

This invention was made with government support under Grant Nos. AI140726 and AI141178, awarded by National Institutes of Health. The government has certain rights in the invention.

### FIELD OF THE INVENTION

The present invention features compositions and methods of use thereof for the treatment of a fungal infection (e.g., cryptococcosis).

### BACKGROUND OF THE INVENTION

Fungal infection, although not very contagious, has affected 300 million people worldwide. With limited classes of available antifungals and chronic treatment, drug resistance is of significant concern. The World Health Organization (WHO) has declared that antimicrobial resistance is one of the top 10 global public health threats facing humanity. Candidiasis, cryptococcosis, aspergillosis, and pneumocystis are major fungal infections. In addition, other fungal diseases, such as histoplasmosis, coccidioidomycosis (valley fever), and blastomycosis, are reported in various parts of the world. Cryptococcosis is caused by opportunistic fungal pathogens Cryptococcus neoformans (Cne) and Cryptococcus gattii (Cga). Immunocompromised individuals are the most affected. Approximately 223,100 human patients are affected by cryptococcosis meningitis globally, with 180,000 deaths annually. Cne and Cga initially affect the lungs and, in severe conditions, the brain, leading to meningitis. AIDS patients with Cryptococcus meningitis are of major concern in developing countries, especially the Sub-Saharan African region.

Amphotericin B (AmB) alone or in combination with 5-flucytosine (5-Fc) or fluconazole (Flu) is used as a frontline therapy in cryptococcal meningitis or severe pulmonary cryptococcosis infections. Reports on drug resistance antifungals are on the rise. An additional class of drugs can help to manage drug resistance. The present invention features compositions (e.g., small molecules) of antifungal activity against Cryptococcus (e.g., Cryptococcus neoformans or Cryptococcus gattii).

### BRIEF SUMMARY OF THE INVENTION

It is an objective of the present invention to provide compositions and methods that allow for the treatment of a fungal infection (e.g., cryptococcosis), as specified in the independent claims. Embodiments of the invention are given in the dependent claims. Embodiments of the present invention can be freely combined with each other if they are not mutually exclusive.

Chronic diseases need prolonged treatment; hence, there is a higher possibility for pathogens to acquire drug resistance, especially when there are only a few classes of drugs available for clinical use. Drug resistance is being reported in many disease treatments, including tuberculosis and cryptococcosis. Various treatment strategies for cryptococcosis are under investigation.

The present invention features a cell-based anti-Cryptococcus neoformans assay to screen inhibitors for cryptococcosis. From the screening, 91 compounds were identified with anti-Cryptococcus neoformans activity with IC₅₀ less than 10 μM. Overall, these compounds represent promising inhibitors for cryptococcosis management.

In some embodiments, the present invention features a composition for use in a method of treating a fungal infection, the composition comprising an antifungal agent in a pharmaceutically acceptable carrier. In other embodiments, the present invention features a method for treating a fungal infection in a subject in need thereof, the method comprising administering a therapeutically effective amount of an antifungal composition to the subject, wherein the antifungal composition comprises an antifungal agent in a pharmaceutically acceptable carrier.

One of the unique and inventive technical features of the present invention is the use of novel antifungals with the potential to treat cryptococcosis. Without wishing to limit the invention to any theory or mechanism, it is believed that the technical feature of the present invention advantageously provides for novel compositions (e.g., small molecules) of antifungal activity against C. neoformans and C. gattii, two of the main causative agents of cryptococcosis. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. In some embodiments, the subject is immunocompromised. In other embodiments, the subject is immunocompetent.

As used herein, the terms “treat,” “treating,” or “treatment” refer to both therapeutic treatment and prophylactic or preventative measures, with the objective of preventing, reducing, slowing down (lessen), inhibiting, or eliminating an undesired physiological change, symptom, disease, or disorder. For example, the disease may be a cryptococcosis infection. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject or patient may be identified (e.g., diagnosed) as one suffering from the disease or condition prior to administration of the compositions of the invention. Subjects at risk for the disease can be identified by, for example, any or a combination of appropriate diagnostic or prognostic assays known in the art.

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

A “therapeutically effective amount” refers to an amount that is sufficient to achieve the desired therapeutic result or to have an effect on undesired symptoms but is generally insufficient to cause intolerable adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As described above, the compositions can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semi-permeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, anti-inflammatory agents, anesthetics, and the like.

The terms “administering” and “administration” refer to methods of providing a pharmaceutical preparation, composition, or formulation to a subject. The compositions described herein can be administered in a number of ways depending on whether local or systemic treatment is desired, and on the area to be treated. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, topically or the like.

Pharmaceutical peptide compositions for oral administration include, but are not limited to, powders or granules, suspensions or solutions in water or non-aqueous media, pills, lozenges, capsules, sachets, or tablets. Thickeners, flavorings, diluents, emulsifiers, dispersing aids or binders may be desirable. A person of skill, monitoring a subject's clinical response, can adjust the frequency of administration and dosage of the medication according to methods known in the art.

For example, the peptide compositions described herein can be administered intranasally or administration by inhalant. As used herein, “intranasal administration” means delivery of the compositions into the nose and nasal passages through one or both of the nares and can comprise delivery by a spraying mechanism (device) or droplet mechanism (device), or through aerosolization of the composition, e.g., by using a nasal spray, atomizer, dropper, or syringe. Administration of the compositions by inhalant can be through the nose or mouth via delivery by a spraying or droplet mechanism. As used herein, “an inhaler” can be a spraying device or a droplet device for delivering the peptide composition, in a pharmaceutically acceptable carrier, to the nasal passages and the upper and/or lower respiratory tracts of a subject. Delivery can also be directly to any area of the respiratory system (e.g., lungs) via intratracheal intubation. A person of skill, monitoring a subject's clinical response, can adjust the frequency of administration and dosage of the medication according to methods known in the art.

According to some embodiments, the present invention features compositions and methods for the treatment of a fungal infection (e.g., cryptococcosis).

The present invention may feature a composition for use in a method of treating a fungal infection, the composition comprising an antifungal agent in a pharmaceutically acceptable carrier. In some embodiments, the present invention features a composition for use in a method of treating and/or preventing a fungal infection caused by a fungus in the Cryptococcus genus, the composition comprising an antifungal agent in a pharmaceutically acceptable carrier. In other embodiments, the present invention features a composition for use in a method of treating and/or preventing a fungal infection caused by Cryptococcus neoformans, the composition comprising an antifungal agent in a pharmaceutically acceptable carrier. In further embodiments, the present invention features a composition for use in a method of treating and/or preventing a fungal infection caused by Cryptococcus gattii, the composition comprising an antifungal agent in a pharmaceutically acceptable carrier.

In some embodiments, the present invention may also feature a composition for use in a method of treating and/or preventing cryptococcosis, the composition comprising an antifungal agent in a pharmaceutically acceptable carrier. In other embodiments, the present invention features a composition for use in a method of treating and/or preventing cryptococcal meningitis, the composition comprising an antifungal agent in a pharmaceutically acceptable carrier.

The present invention may also feature a method for treating a fungal infection in a subject in need thereof, the method comprising administering a therapeutically effective amount of an antifungal composition to the subject, wherein the antifungal composition comprises an antifungal agent in a pharmaceutically acceptable carrier. In some embodiments, the present invention features a method for preventing and/or treating a fungal infection caused by a fungus in the Cryptococcus genus in a subject in need thereof, the method comprising administering a therapeutically effective amount of an antifungal composition to the subject, wherein the antifungal composition comprises an antifungal agent in a pharmaceutically acceptable carrier. In other embodiments, the present invention features a method for preventing and/or treating a fungal infection caused by Cryptococcus neoformans in a subject in need thereof, the method comprising administering a therapeutically effective amount of an antifungal composition to the subject, wherein the antifungal composition comprises an antifungal agent in a pharmaceutically acceptable carrier. In further embodiments, the present invention features a method for preventing and/or treating a fungal infection caused by Cryptococcus gattii in a subject in need thereof, the method comprising administering a therapeutically effective amount of an antifungal composition to the subject, wherein the antifungal composition comprises an antifungal agent in a pharmaceutically acceptable carrier.

In some embodiments, the present invention may also feature a method of preventing and/or treating cryptococcosis in a subject in need thereof, the method comprising administering a therapeutically effective amount of an antifungal composition to the subject, wherein the antifungal composition comprises an antifungal agent in a pharmaceutically acceptable carrier. In other embodiments, the present invention features a method of preventing and/or treating cryptococcal meningitis in a subject in need thereof, the method comprising administering a therapeutically effective amount of an antifungal composition to the subject, wherein the antifungal composition comprises an antifungal agent in a pharmaceutically acceptable carrier.

The aforementioned antifungal agents (e.g., the antifungal composition) may be selected from a group consisting of N-[2-(4-chlorophenyl)ethyl]thieno[2,3-d]pyrimidin-4-amine, 4-benzyl-2,3,4,5,7,8,9,10-octahydrospiro[1,4-benzoxazacyclododecine-6,4″-piperidine], 6-(2,3-Dihydro-1,4-Benzodioxin-6-Yl)-3-[(4-Methoxyphenoxy)Methyl][1,2,4]Triazolo[3,4-B][1,3,4]Th iadiazole, NH1125, 6-(4-Me6-(4-Methylbenzyl)-2-Phenyl[1,2,4]Triazolo[1,5-C]Quinazolin-5 (6H)-One, haloprogin, AC-93253 iodide (2-[3-(1,3-Dihydro-1,3,3-trimethyl-2H-indol-2-ylidene)-1-propenyl]-3-ethyl-benzothiazolium iodide), C18H18ClN5O, 3-[[4-[2-(3-chloroanilino) pyrimidin-4-yl]pyridin-2-yl]amino]propan-1-ol, N-(3-Chloro-4-Methylphenyl)-N′-(1-Methyl-5-Phenyl-1H-Imidazol-2-Yl)Urea, QNZ, 1-benzyl-4-[(4-fluorophenyl)methylsulfanyl]-6,7-dihydro-5-cyclopenta[d]pyrimidin-2-one, 1-(3,6-dibromocarbazol-9-yl)-3-(3-methylbutylamino)propan-2-ol, CD 1530, Tetrindole mesylate, N-[3-(4-benzylpiperidin-1-yl)propyl]-9-methyl-4H-thieno [3,2-c]chromene-2-carboxamide, MCULE-2046798638, NVP-TAE 226, Calmidazolium chloride, R 24571 chloride, 3,6-dimethyl-N-[(4-methylphenyl)methyl]-[1,2]oxazolo[5,4-d]pyrimidin-4-amine, N-[2-(cyclohexen-1-yl)ethyl]-3,6-dimethyl-[1,2]oxazolo[5,4-d]pyrimidin-4-amine, 1-(3-chloro-2-methylphenyl)-1H-pyrrole-2,5-dione, 1-benzyl-4-benzylsulfanyl-6,7-dihydro-5H-cyclopenta[d]pyrimidin-2-one, CBR 5884, N-[2-(cyclohexen-1-yl)ethyl]-3-ethyl-6-methyl-[1,2]oxazolo[5,4-d]pyrimidin-4-amine, 6-(3-chloro-4-methylphenyl)-5-(2-fluorophenyl)-[1,2,5]oxadiazolo[3,4-b]pyrazine-5,6-diamine, PluriSIn #1 (NSC 14613), 1-(9-ethylcarbazol-3-yl)pyrrole-2,5-dione, 1-(4-Butyl-phenyl)-pyrrole-2,5-dione, Thonzonium bromide, HSP990 (NVP-HSP990), 2-[3-(3-methylbutoxy)phenoxy]-N-pyridin-4-ylacetamide, N-[2-(cyclohexen-1-yl)ethyl]-3-ethyl-[1,2]oxazolo[5,4-d]pyrimidin-4-amine, 1-(2-bromophenyl)-1H-pyrrole-2,5-dione, Adaptaquin, JAK3 Inhibitor VI, Fingolimod, Cyto4A5, K-252a (Nocardiopsis sp.), N-[4-bromo-3-(trifluoromethyl)phenyl]thiophene-2-carboxamide, BAY 11-7082, Methiazole, 2-AAPA hydrate, 1-(3,5-Dimethylphenyl)-1H-pyrrole-2,5-dione, MNS (3,4-Methylenedioxy-p-nitrostyrene, MDBN), 1-methyl-N~6~~(2-methylphenyl)-N~4~~phenyl-1H-pyrazolo[3,4-d]pyrimidine-4,6-diamine, STK890868, 1-(4-butan-2-ylphenyl)pyrrole-2,5-dione, Benzethonium chloride, 6-(3,4-dimethoxyphenyl)-3-[(4-methoxyphenoxy)methyl]-[1,2,4]triazolo[3,4-b][1,3,4]thiadiazole, Maleimide (N-phenethyl-N-(2-phenylethyl)thieno[3,2-d]pyrimidin-4-amine), 5-Nonyloxytryptamine, 2-[[5-(2,4-dichlorophenyl)furan-2-yl]methylamino]-1-phenylethanol, AS 1517499, 3-methyl-4-(3-phenylpyrrolidin-1-yl)-[1,2]oxazolo[5,4-d]pyrimidine, BADGE, 1-(1,3-benzodioxol-5-yl)-N-[[5-(4-chlorophenyl)furan-2-yl]methyl]methanamine, Ethyl 1-(5-methylthieno[2,3-d]pyrimidin-4-yl)piperidine-4-carboxylate, Keratinocyte Differentiation Inducer, N-[(4-chlorophenyl)methyl]-1-(5,6-dimethylfuro [2,3-d]pyrimidin-4-yl)piperidine-4-carboxamide, 1-(5-Chloro-2-methylphenyl)-4-[(1-{[2-(4-chlorophenyl)-5-methyl-1,3-oxazol-4-yl]methyl}piperidin-4-yl)carbonyl]piperazine, 1-[4-[3-(trifluoromethyl)pyrazol-1-yl]phenyl]pyrrole-2,5-dione, 1-[4-(61-[4-(6-methyl-1,3-benzothiazol-2-yl)phenyl]-3-(4-methylpiperidin-1-yl)pyrrolidine-2,5-dion, 1,3-dihydroxyxanthen-9-one, N-(3-chN-(3-chloro-4-fluorophenyl)-4-[[2-(4-fluorophenyl)pyrimidin-4-yl]amino]benzamide, 6-chloro-12-[[4-[2-(dimethylamino)ethoxy]-3-methoxyphenyl]methyl]-2,19-dioxa-9,12-diazatricyclo[18.4.0.03,8]tetracosa-1(24),3(8),4,6,20,22-hexaen-10-one, Tyrphostin 9, 2-(6-chloro-2,4-dihydropyrido[3,2-h][1,3]benzoxazin-3-yl)-N,N-diethylethanamine, 1-((4-Chlorophenyl)methyl)-1h-pyrrole-2,5-dione, 1-(1,3-benzodioxol-5-yl)-N-[[5-(4-bromophenyl)furan-2-yl]methyl]methanamine, 2-(4-butoxyphenyl)-4-(4-ethylpiperazin-1-yl)pyrazolo[1,5-a]pyrazine, 1-[3-(Methylsulfanyl)phenyl]-2,5-dihydro-1H-pyrrole-2,5-dione, 1-(4-bromophenyl)-3-[2-(diethylamino)-4-methylquinolin-6-yl]urea, N-[[5-(2,4-dichlorophenyl)furan-2-yl]methyl]-1-pyridin-2-ylmethanamine, Lylamine hydrochloride, 4,6-Diphenyl-1-hydroxy-pyridine-2-one, 8-(4-fluorophenyl)-2-(4-methoxyphenyl)-N-(2-piperidin-1-ylethyl)quinoline-4-carboxamide MCULE-4343682937, 5-(4-methylphenyl)sulfanylquinazoline-2,4-diamine, 2-chloro-4-(4-methylpiperidin-1-yl)quinazoline, 6-Phenoxybenzo[d]thiazol-2-amine, 3-[(Dimethylamino) methyl]-2,3-dihydrochromen-4-one;chloride, 1-[2-(2,4-dichlorophenoxy)phenyl]pyrrole-2,5-dione, Anisomycin, 2-[[5-(2,5-dichlorophenyl)furan-2-yl]methylamino]-1-phenylethanol, N-[5-tert-butyl-3-(4-fluorophenyl)-2-methylpyrazolo[1,5-a]pyrimidin-7-yl]-N′,N′-dimethylethan-1,2-diamine, Staurosporine, N-[[5-(4-bromophenyl)furan-2-yl]methyl]-1-(furan-2-yl) methanamine, N-benzyl-3,6-dimethyl-[1,2]oxazolo[5,4-d]pyrimidin-4-amine, 4-benzyl-1″-(1-methylpiperidine-4-carbonyl)-2,3,4,5,7,8,9,10-octahydrospiro[1,4-benzoxazacyclododecine-6,4″-piperidine], LE 135, AZ-960, N,N-diphenyl-4-[(E)-1,2,4-triazol-4-yliminomethyl]aniline, and N-{[2-(4-ethylphenyl)imidazo[1,2-a]pyridin-3-yl]methyl}-N-(3-methylbenzyl)amine (MCULE-1645297855) (See Table 1 and chemical structures).

In other embodiments, the antifungal agents (e.g., the antifungal composition) may be selected from a group consisting of: N-[2-(4-chlorophenyl)ethyl]thieno[2,3-d]pyrimidin-4-amine, 4-benzyl-2,3,4,5,7,8,9,10-octahydrospiro[1,4-benzoxazacyclododecine-6,4″-piperidine], 6-(2,3-Dihydro-1,4-Benzodioxin-6-Yl)-3-[(4-Methoxyphenoxy)Methyl][1,2,4]Triazolo[3,4-B][1,3,4]Th iadiazole, NH1125, 6-(4-Me6-(4-Methylbenzyl)-2-Phenyl[1,2,4][1,5-C]Quinazolin-5(6H)-One, Haloprogin, AC-93253 iodide, 2-[3-(1,3-Dihydro-1,3,3-trimethyl-2H-indol-2-ylidene)-1-propenyl]-3-ethyl-benzothiazolium iodide, C18H18ClN5O, 3-[[4-[2-(3-chloroanilino)pyrimidin-4-yl]pyridin-2-yl]amino]propan-1-ol, N-(3-Chloro-4-Methylphenyl)-N′-(1-Methyl-5-Phenyl-1H-Imidazol-2-Yl)Urea, QNZ, 1-benzyl-4-[(4-fluorophenyl)methylsulfanyl]-6,7-dihydro-5-cyclopenta[d]pyrimidin-2-one, 1-(3,6-dibromocarbazol-9-yl)-3-(3-methylbutylamino)propan-2-ol, CD 1530, Tetrindole mesylate, N-[3-(4-benzylpiperidin-1-yl)propyl]-9-methyl-4H-thieno [3,2-c]chromene-2-carboxamide, MCULE-2046798638, NVP-TAE 226, Calmidazolium chloride, R 24571 chloride, 3,6-dimethyl-N-[(4-methylphenyl)methyl]-[1,2]oxazolo[5,4-d]pyrimidin-4-amine, N-[2-(cyclohexen-1-yl)ethyl]-3,6-dimethyl-[1,2]oxazolo[5,4-d]pyrimidin-4-amine, 1-(3-chloro-2-methylphenyl)-1H-pyrrole-2,5-dione, 1-benzyl-4-benzylsulfanyl-6,7-dihydro-5H-cyclopenta[d]pyrimidin-2-one, CBR 5884, N-[2-(cyclohexen-1-yl)ethyl]-3-ethyl-6-methyl-[1,2]oxazolo[5,4-d]pyrimidin-4-amine, 6-(3-chloro-4-methylphenyl)-5-(2-fluorophenyl)-[1,2,5]oxadiazolo[3,4-b]pyrazine-5,6-diamine, PluriSIn #1 (NSC 14613), 1-(9-ethylcarbazol-3-yl)pyrrole-2,5-dione, 1-(4-Butyl-phenyl)-pyrrole-2,5-dione, Thonzonium bromide, HSP990 (NVP-HSP990), 2-[3-(3-methylbutoxy)phenoxy]-N-pyridin-4-ylacetamide, N-[2-(cyclohexen-1-yl)ethyl]-3-ethyl-[1,2]oxazolo[5,4-d]pyrimidin-4-amine, 1-(2-bromophenyl)-1H-pyrrole-2,5-dione, Adaptaquin, JAK3 Inhibitor VI, Fingolimod, Cyto4A5, K-252a, Nocardiopsis sp., and N-[4-bromo-3-(trifluoromethyl)phenyl]thiophene-2-carboxamide.

In some embodiments, the fungal infection is caused by a fungus in the genus Cryptococcus. In some embodiments, the fungus is Cryptococcus neoformans. In some embodiments, the fungus is Cryptococcus gattii. In some embodiments, the fungal infection is caused by Cryptococcus neoformans. In some embodiments, the fungal infection is caused by Cryptococcus gattii.

In some embodiments, the fungal infection comprises cryptococcus. In some embodiments, the fungal infection is cryptococcal meningitis.

### Example 1

The following is a non-limiting example of the present invention. It is to be understood that said example is not intended to limit the present invention in any way. Equivalents or substitutes are within the scope of the present invention.

For primary screening, a cell-based high-throughput screening (HTS) assay was developed for testing the growth inhibition of Cne H99 strain of fungus. Conversion of non-fluorescent resazurin to fluorescent resofurin by fungal aerobic respiration of metabolically active cells was used to estimate the fungicidal or fungistatic activity of test compounds. After standardizing the assay in 96 well plates, it was optimized in a 384-well plate.

The compound library included known bioactives, commercial libraries, and academic molecules. For HTS, the fungal cells were grown in SD broth overnight and diluted in Sabouraud dextrose (SD) broth to an optical density at 600 nm (OD600) of 0.5. The cell suspension was further diluted 1:10 in Roswell Park Memorial Institute Medium (RPMI) 1640 medium with 0.165 M MOPS (3-(N-morpholino)propanesulfonic acid) and 2% glucose (RPMI-MOPS) so that the final OD will be 0.05. From this cell suspension, 20 μL of the cells was added to each well of the 384-well assay plate (black, clear bottom) containing test compounds. Plates with test compounds were prepared by adding 20 μL of RPMI-MOPS medium to each well using a liquid dispenser (Thermo Multidrop Combi), followed by transferring 1 μL of the test compounds to all wells of the 22 columns in the assay plate using a robot. Dimethyl sulfoxide (DMSO) as vehicle control or 0.8 μM AmB as positive control was added manually to the remaining two columns using a multichannel pipet. After addition of fungal cells, the plates were incubated in a humidified incubator at 30° C. for 24 h. A 10× stock solution of resazurin in phosphate-buffered saline (PBS) was diluted in PBS and RPMI-MOPS to 1×. Resazurin (10 μL) was added to each well to a final concentration of 44 μM using a liquid dispenser (Multidrop Combi, ThermoFisher Scientific) and further incubated at 30° C. overnight. The conversion of resazurin to resorufin by live cells was estimated by reading fluorescence at Ex 550 nm and Em 590 nm using a plate reader (EnVision, PerkinElmer). Additionally, the absorbance was also read at 600 nm using the plate reader (EnVision, PerkinElmer).

Active compounds that showed at least 50% inhibition in the primary assay were selected for dose-response assay to determine the IC₅₀. Concentration series (0.02-50 μM) of test compounds and equalizing volume of DMSO were dispensed, using a liquid handler (D300e, Hewlett-Packard), to the 384-well plates. Following the addition of the Cne cells, the assay plates were incubated for 24 hours, followed by a fluorescence reading of resorufin as explained for single-concentration screening. The IC₅₀ was calculated by graphpad prism.

Below are non-limiting examples of compounds (e.g., antifungal agents) with antifungal (e.g., anti-Cryptococcus neoformans) activity.

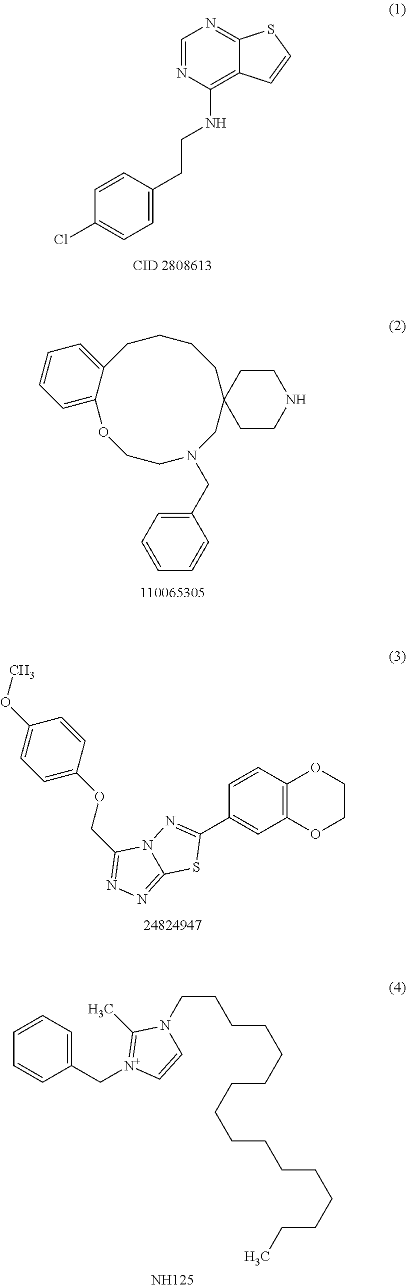 [SMILES: Clc1ccc(CCNc2ncnc3sccc23)cc1 ; c1ccc(CN2CCOc3ccccc3CCCCC3(CCNCC3)C2)cc1 ; COc1ccc(OCc2nnc3sc(-c4ccc5c(c4)OCCO5)nn23)cc1 ; CCCCCCCCCCCCCCCCn1cc[n+](Cc2ccccc2)c1C ; "NH125"]

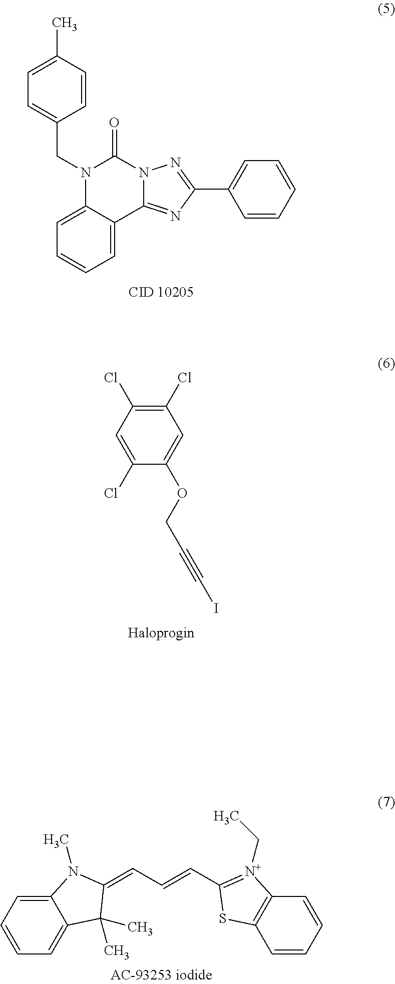 [SMILES: Cc1ccc(Cn2c(=O)n3nc(-c4ccccc4)nc3c3ccccc32)cc1 ; Clc1cc(Cl)c(OCC#CI)cc1Cl ; CC[n+]1c(/C=C/C=C2/N(C)c3ccccc3C2(C)C)sc2ccccc21]

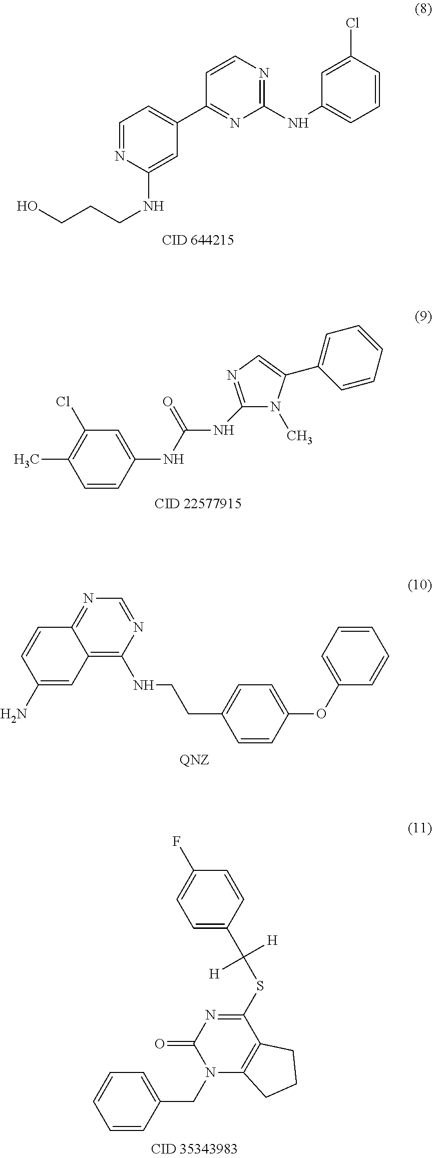 [SMILES: OCCCNc1cc(-c2ccnc(Nc3cccc(Cl)c3)n2)ccn1 ; Cc1ccc(NC(=O)Nc2ncc(-c3ccccc3)n2C)cc1Cl ; Nc1ccc2ncnc(NCCc3ccc(Oc4ccccc4)cc3)c2c1 ; O=c1nc(SCc2ccc(F)cc2)c2c(n1Cc1ccccc1)CCC2]

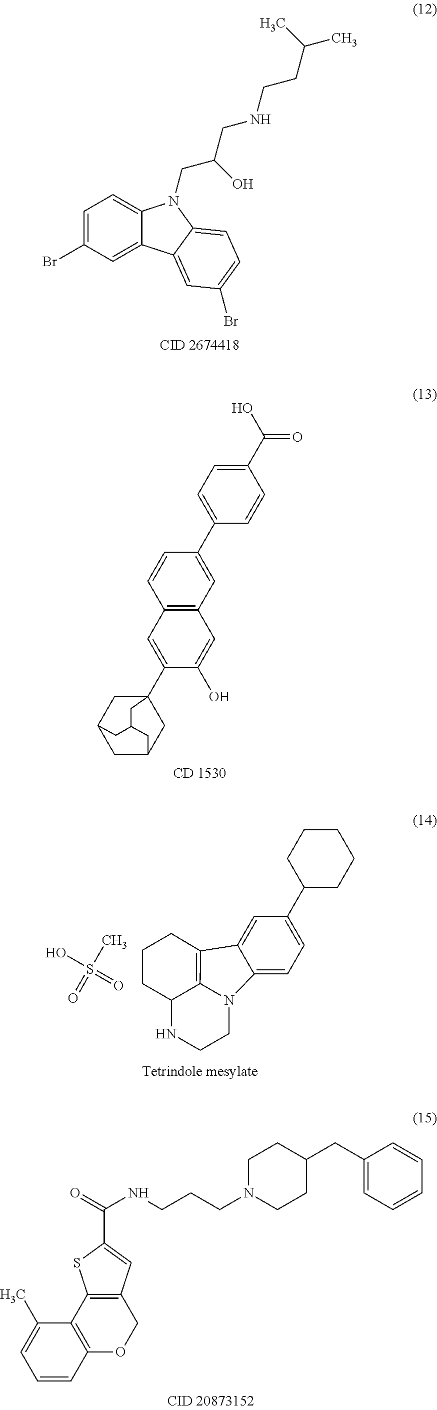

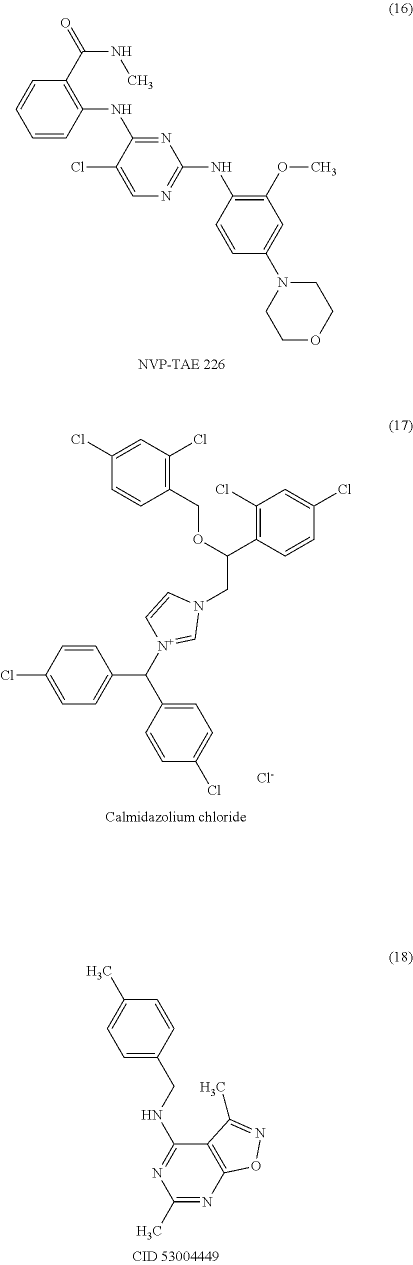 [SMILES: CNC(=O)c1ccccc1Nc1nc(Nc2ccc(N3CCOCC3)cc2OC)ncc1Cl ; Clc1ccc(C(c2ccc(Cl)cc2)[n+]2ccn(CC(OCc3ccc(Cl)cc3Cl)c3ccc(Cl)cc3Cl)c2)cc1 ; [Cl-] ; Cc1ccc(CNc2nc(C)nc3onc(C)c23)cc1]

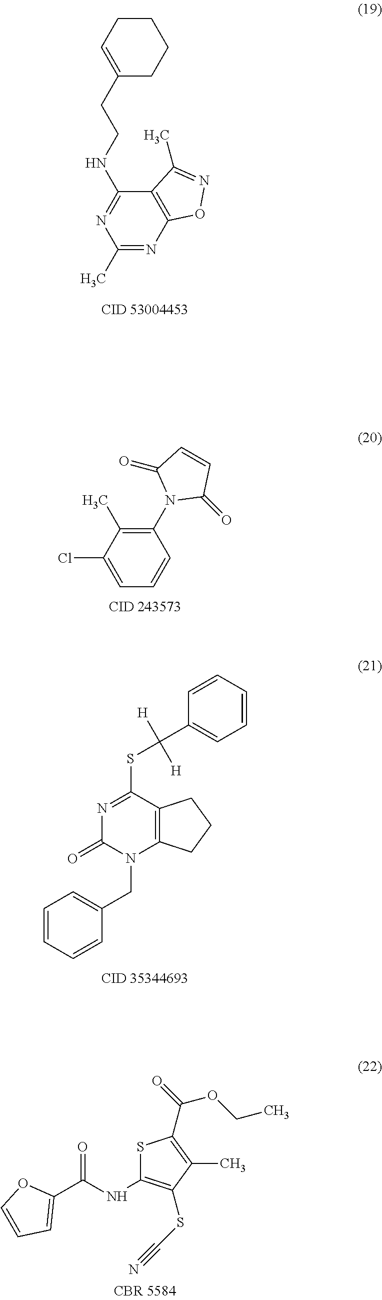 [SMILES: Cc1nc(NCCC2=CCCCC2)c2c(C)noc2n1 ; Cc1c(Cl)cccc1N1C(=O)C=CC1=O ; O=c1nc(SCc2ccccc2)c2c(n1Cc1ccccc1)CCC2 ; CCOC(=O)c1sc(NC(=O)c2ccco2)c(SC#N)c1C]

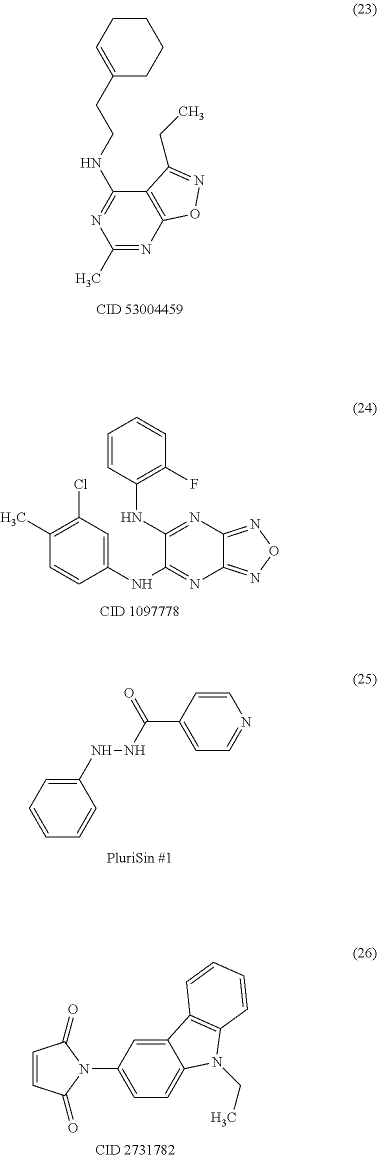 [SMILES: CCc1noc2nc(C)nc(NCCC3=CCCCC3)c12 ; Cc1ccc(Nc2nc3nonc3nc2Nc2ccccc2F)cc1Cl ; O=C(NNc1ccccc1)c1ccncc1 ; CCn1c2ccccc2c2cc(N3C(=O)C=CC3=O)ccc21]

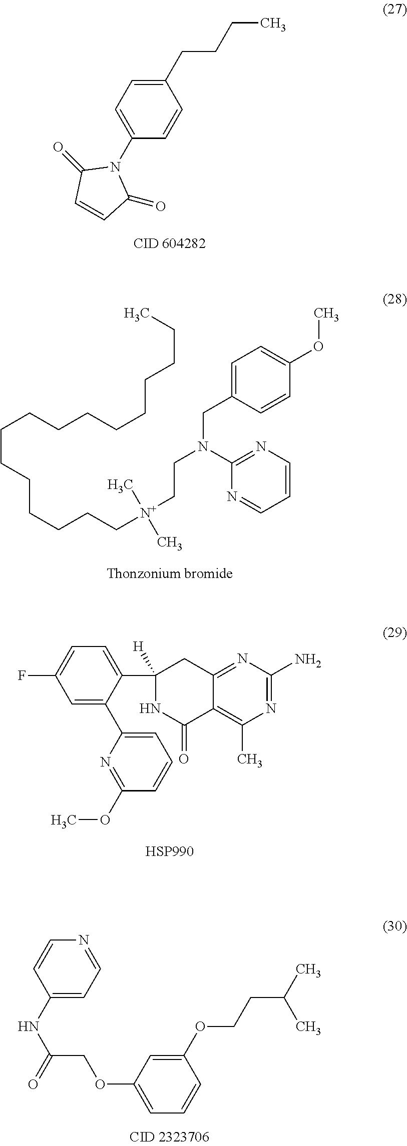 [SMILES: CCCCc1ccc(N2C(=O)C=CC2=O)cc1 ; CCCCCCCCCCCCCCCC[N+](C)(C)CCN(Cc1ccc(OC)cc1)c1ncccn1 ; COc1cccc(-c2cc(F)ccc2[C@H]2Cc3nc(N)nc(C)c3C(=O)N2)n1 ; "HSP990" ; CC(C)CCOc1cccc(OCC(=O)Nc2ccncc2)c1]

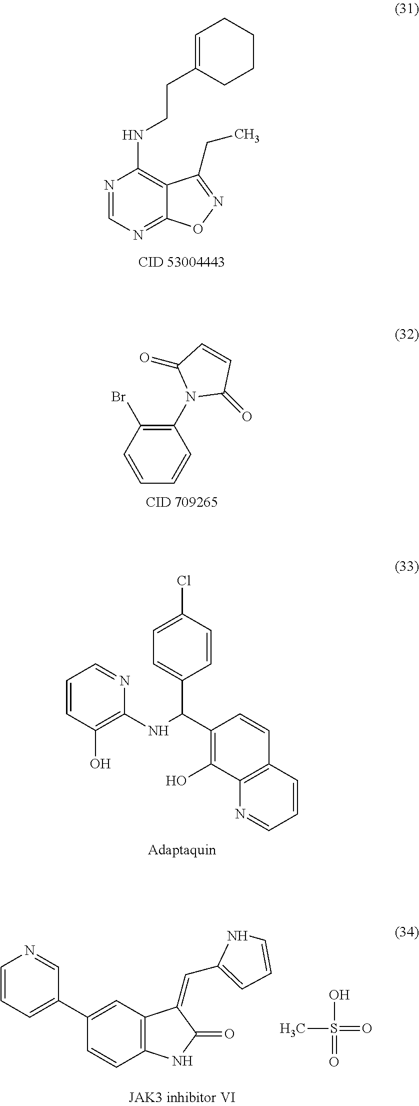

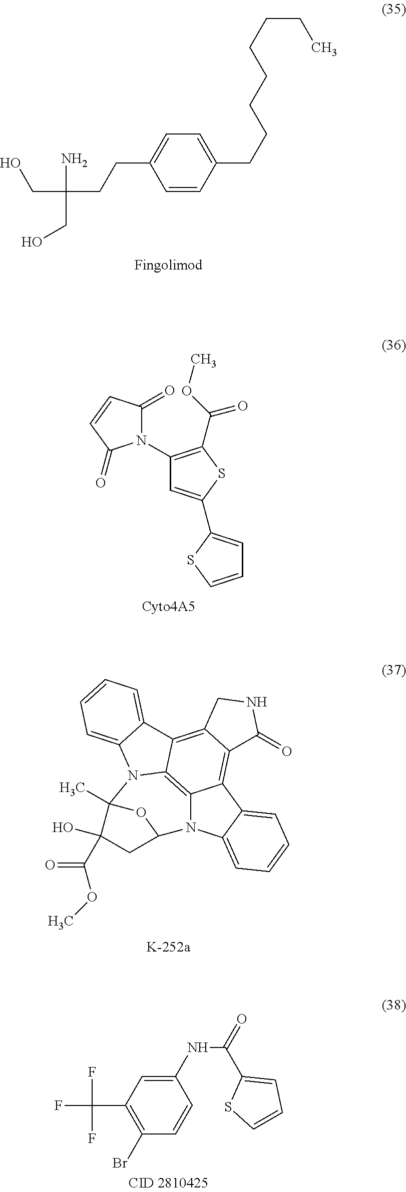 [SMILES: CCCCCCCCc1ccc(CCC(N)(CO)CO)cc1 ; COC(=O)c1sc(-c2cccs2)cc1N1C(=O)C=CC1=O ; COC(=O)C1(O)CC2OC1(C)n1c3ccccc3c3c4c(c5c6ccccc6n2c5c31)C(=O)NC4 ; O=C(Nc1ccc(Br)c(C(F)(F)F)c1)c1cccs1]

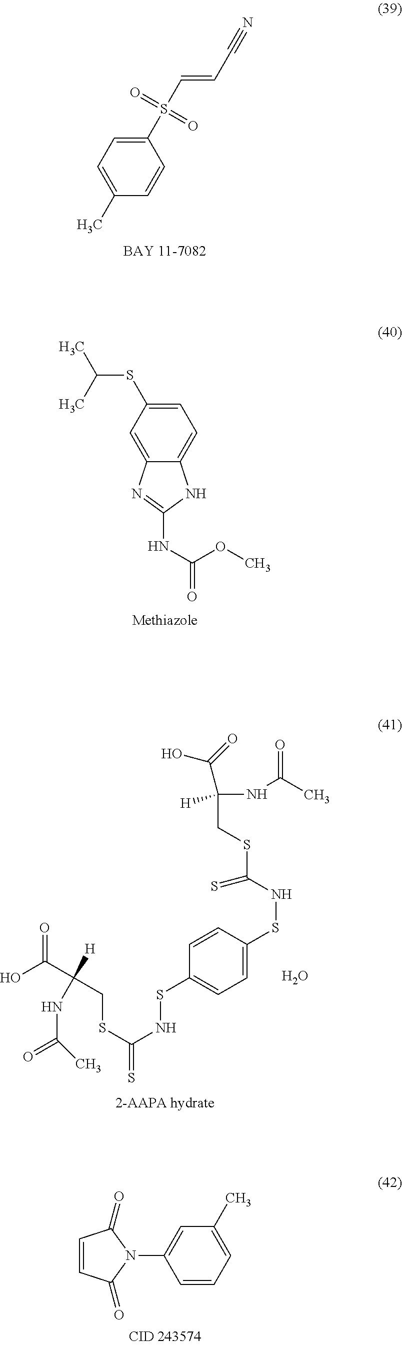

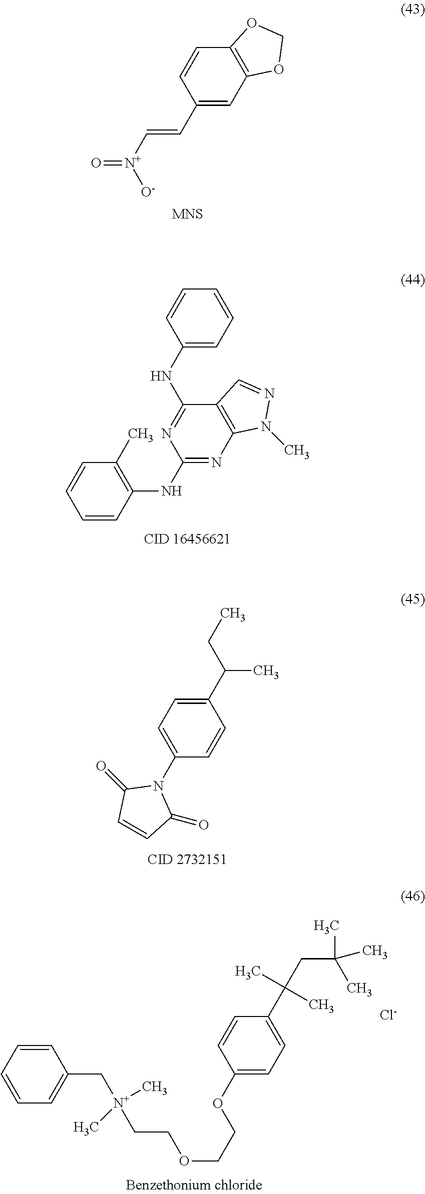

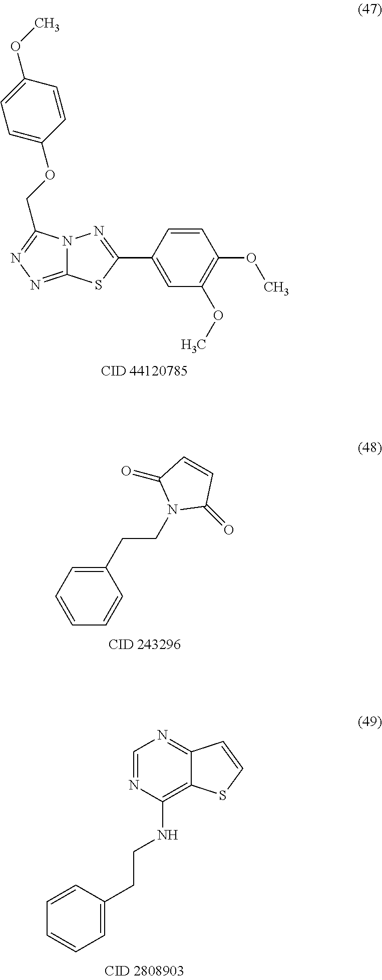 [SMILES: COc1ccc(OCc2nnc3sc(-c4ccc(OC)c(OC)c4)nn23)cc1 ; O=C1C=CC(=O)N1CCc1ccccc1 ; c1ccc(CCNc2ncnc3ccsc23)cc1]

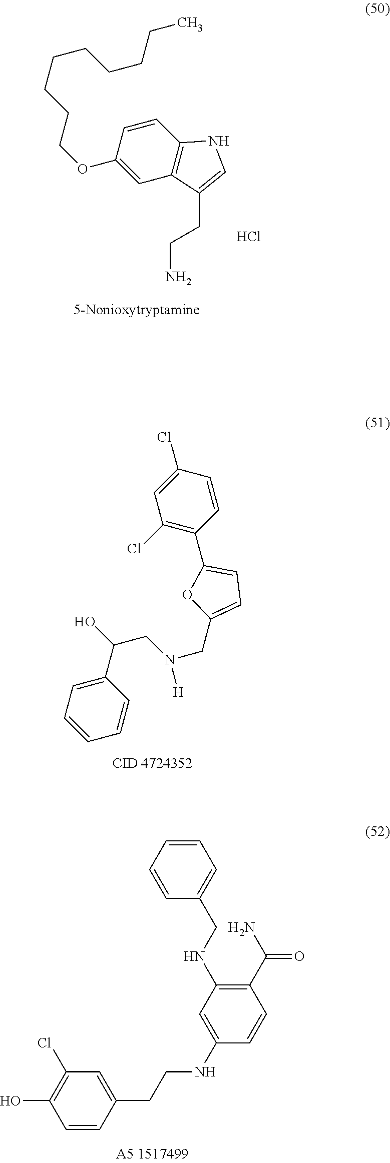 [SMILES: CCCCCCCCCOc1ccc2[nH]cc(CCN)c2c1 ; Cl ; OC(CNCc1ccc(-c2ccc(Cl)cc2Cl)o1)c1ccccc1 ; NC(=O)c1ccc(NCCc2ccc(O)c(Cl)c2)cc1NCc1ccccc1]

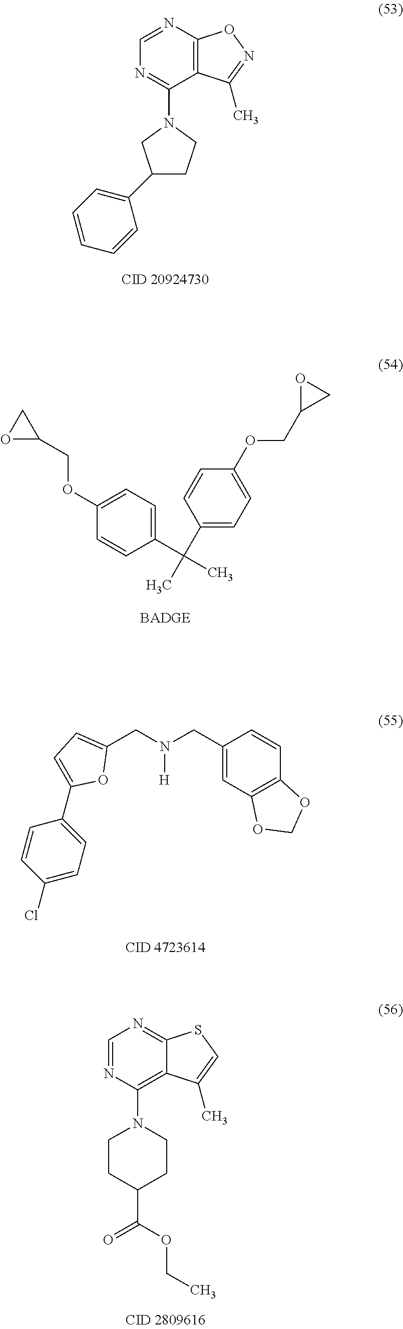 [SMILES: Cc1noc2ncnc(N3CCC(c4ccccc4)C3)c12 ; CC(C)(c1ccc(OCC2CO2)cc1)c1ccc(OCC2CO2)cc1 ; Clc1ccc(-c2ccc(CNCc3ccc4c(c3)OCO4)o2)cc1 ; CCOC(=O)C1CCN(c2ncnc3scc(C)c23)CC1]

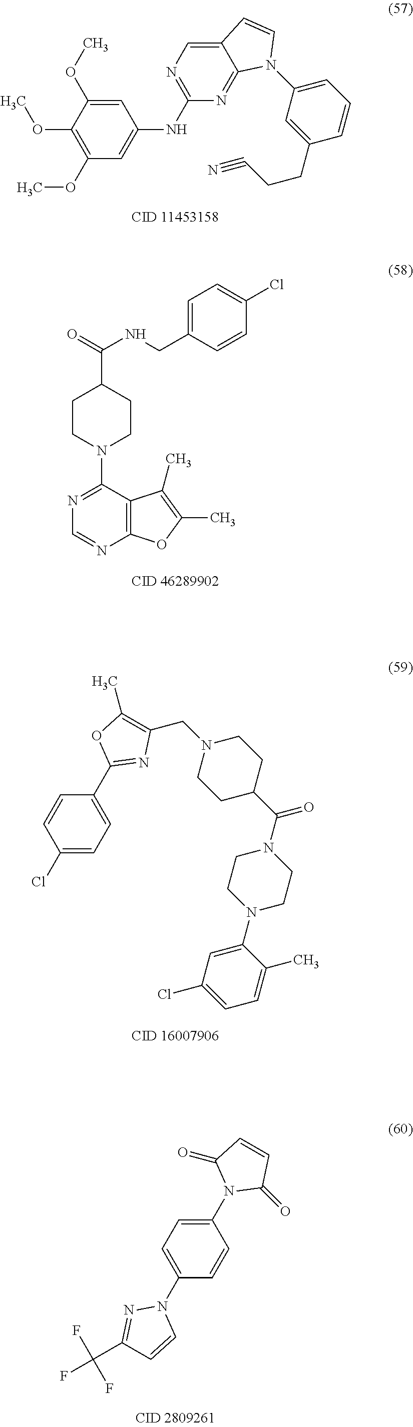 [SMILES: COc1cc(Nc2ncc3ccn(-c4cccc(CCC#N)c4)c3n2)cc(OC)c1OC ; Cc1oc2ncnc(N3CCC(C(=O)NCc4ccc(Cl)cc4)CC3)c2c1C ; Cc1ccc(Cl)cc1N1CCN(C(=O)C2CCN(Cc3nc(-c4ccc(Cl)cc4)oc3C)CC2)CC1 ; O=C1C=CC(=O)N1c1ccc(-n2ccc(C(F)(F)F)n2)cc1]

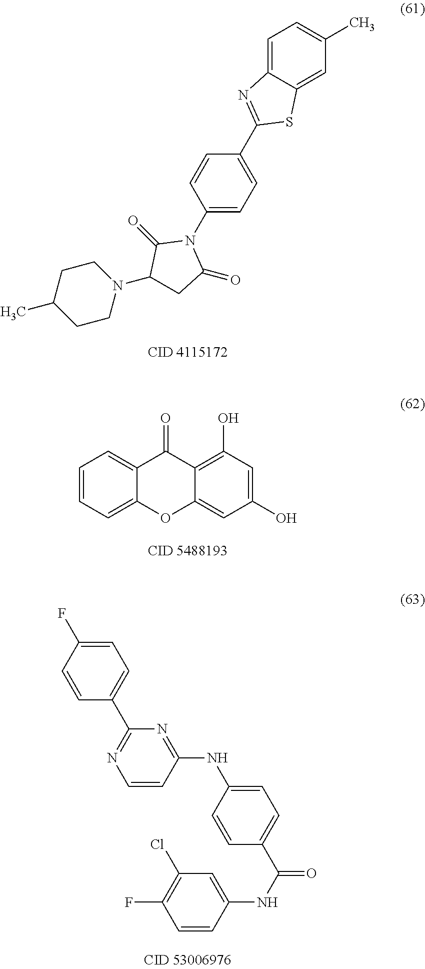

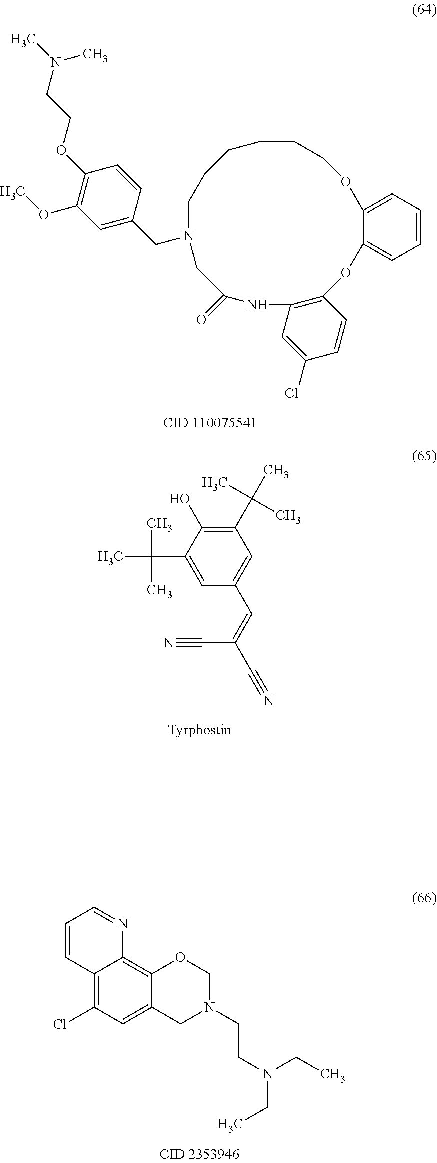 [SMILES: COc1cc(CN2CCCCCCOc3ccccc3Oc3ccc(Cl)cc3NC(=O)C2)ccc1OCCN(C)C ; CC(C)(C)c1cc(C=C(C#N)C#N)cc(C(C)(C)C)c1O ; CCN(CC)CCN1COc2c(cc(Cl)c3cccnc23)C1]

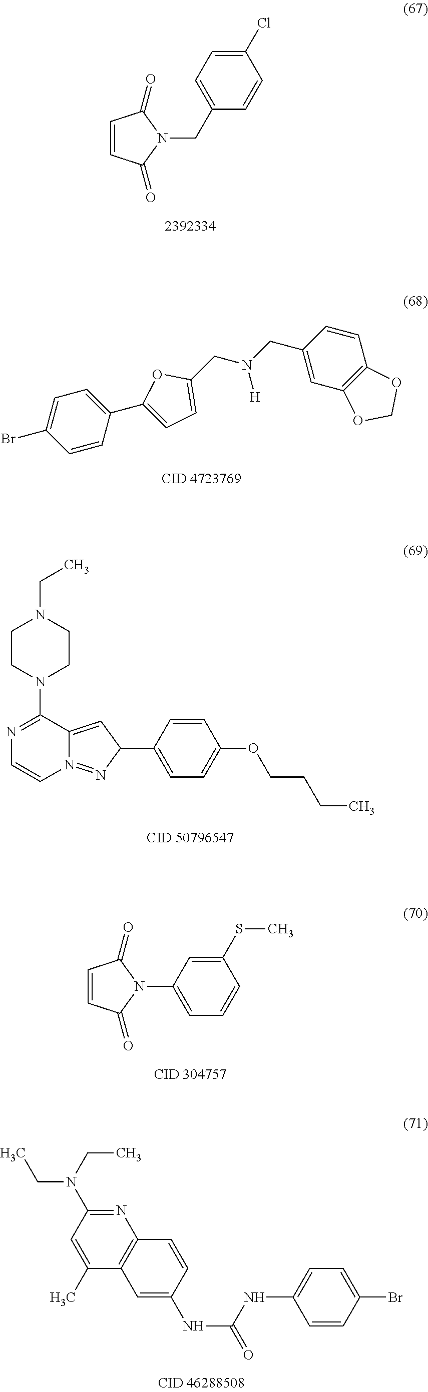

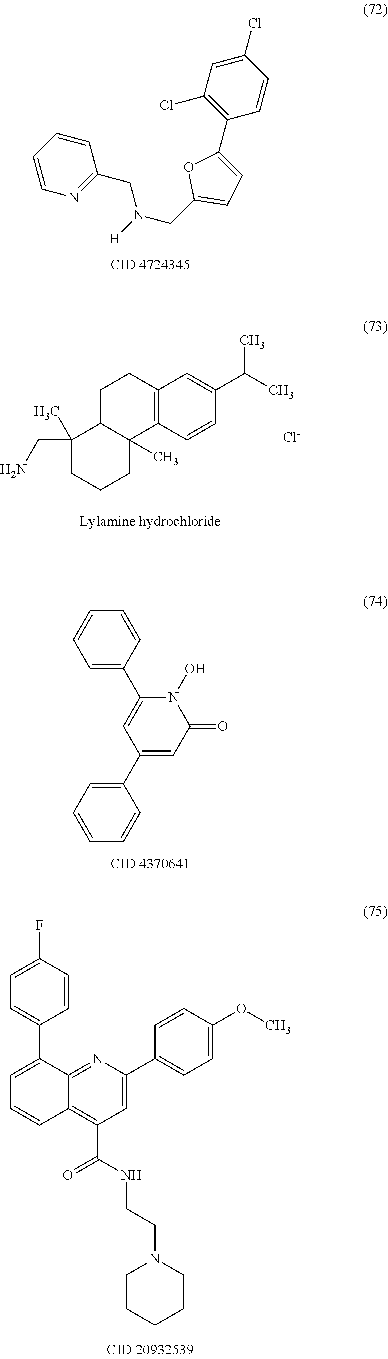 [SMILES: Clc1ccc(-c2ccc(CNCc3ccccn3)o2)c(Cl)c1 ; CC(C)c1ccc2c(c1)CCC1C(C)(CN)CCCC21C ; [Cl-] ; O=c1cc(-c2ccccc2)cc(-c2ccccc2)n1O ; COc1ccc(-c2cc(C(=O)NCCN3CCCCC3)c3cccc(-c4ccc(F)cc4)c3n2)cc1]

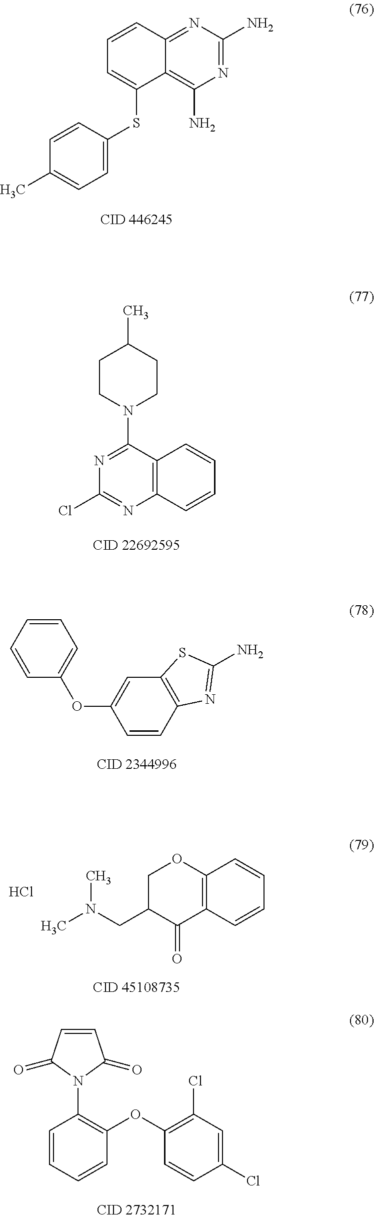 [SMILES: Cc1ccc(Sc2cccc3nc(N)nc(N)c23)cc1 ; CC1CCN(c2nc(Cl)nc3ccccc23)CC1 ; Nc1nc2ccc(Oc3ccccc3)cc2s1 ; Cl ; CN(C)CC1COc2ccccc2C1=O ; O=C1C=CC(=O)N1c1ccccc1Oc1ccc(Cl)cc1Cl]

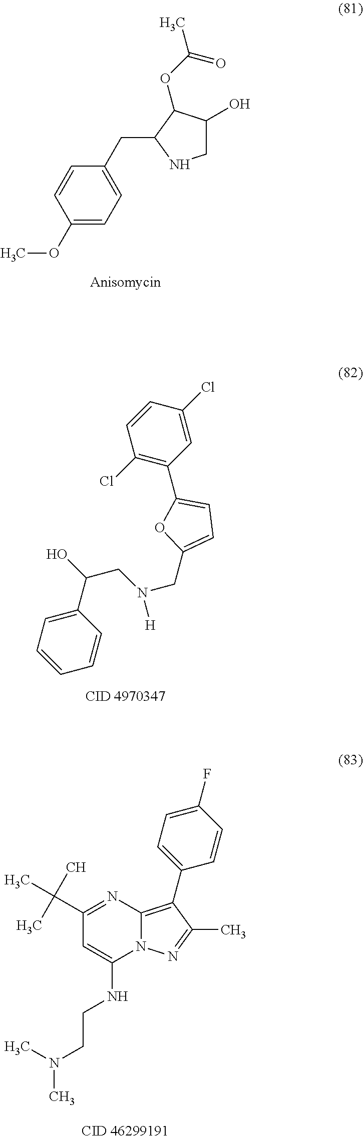 [SMILES: COc1ccc(CC2NCC(O)C2OC(C)=O)cc1 ; OC(CNCc1ccc(-c2cc(Cl)ccc2Cl)o1)c1ccccc1 ; Cc1nn2c(NCCN(C)C)cc(C(C)(C)C)nc2c1-c1ccc(F)cc1]

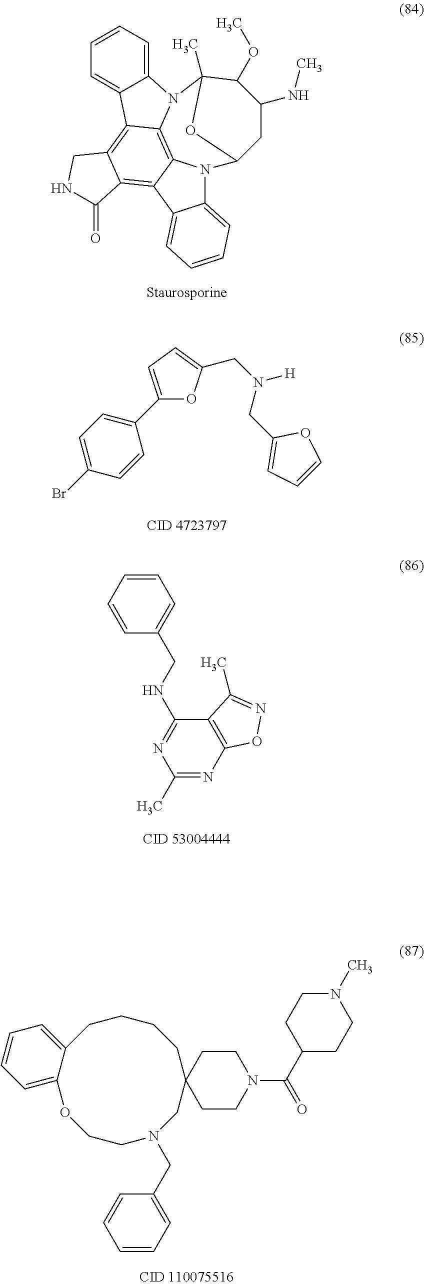 [SMILES: CNC1CC2OC(C)(C1OC)n1c3ccccc3c3c4c(c5c6ccccc6n2c5c31)C(=O)NC4 ; Brc1ccc(-c2ccc(CNCc3ccco3)o2)cc1 ; Cc1nc(NCc2ccccc2)c2c(C)noc2n1 ; CN1CCC(C(=O)N2CCC3(CCCCc4ccccc4OCCN(Cc4ccccc4)C3)CC2)CC1]

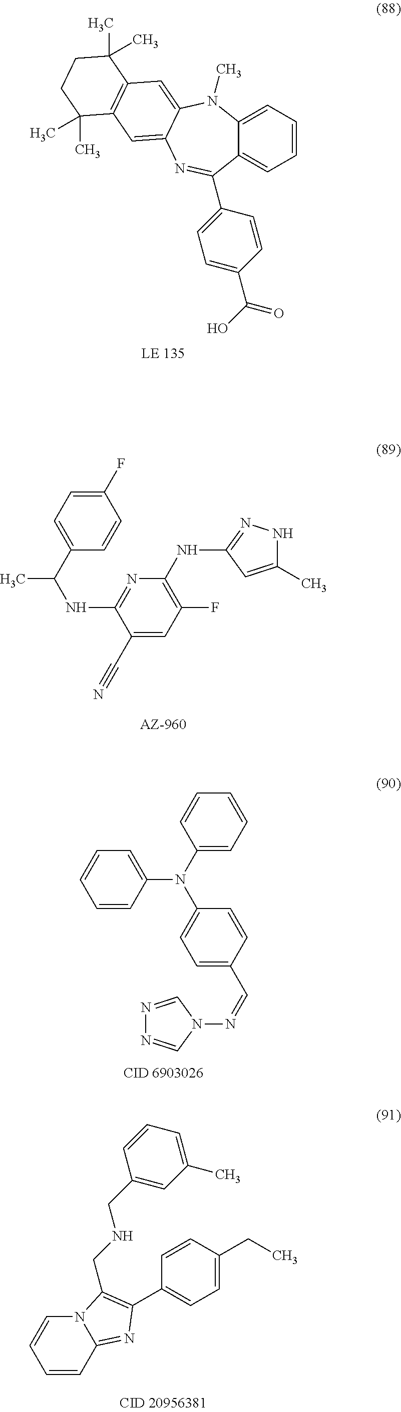 [SMILES: CN1c2cc3c(cc2N=C(c2ccc(C(=O)O)cc2)c2ccccc21)C(C)(C)CCC3(C)C ; Cc1cc(Nc2nc(NC(C)c3ccc(F)cc3)c(C#N)cc2F)n[nH]1 ; C(=N/n1cnnc1)/c1ccc(N(c2ccccc2)c2ccccc2)cc1 ; CCc1ccc(-c2nc3ccccn3c2CNCc2cccc(C)c2)cc1]

A total of 98,441 unique compounds from 36 libraries were screened in the primary assay. From these, 391 compounds were selected for dose-response activity. Compounds that show IC₅₀ less than 10 μM were selected (Table 1). Ninety-one compounds show IC₅₀ less than 10 μM.

TABLE 1
Compounds with anti-Cryptococcus neoformans activity.

| | | | | | Activity |
|---|---|---|---|---|---|
| Sl | | PubChem | | Molecular | in Cne |
| No: | Name | CIDs: | SMILE | Weight | IC₅₀ uM |
| 1 | N-[2-(4-chlorophenyl)ethyl]thieno[2,3-d]pyrimidin-4-amine | 2808613 | Clc1ccc(CCNc2ncnc3sccc23)cc1 | 289.783 | 0.35 |
| 2 | 4-benzyl-2,3,4,5,7,8,9,10-octahydrospiro[1,4-benzoxazacyclododecine-6,4″-piperidine] | 110065305 | C1CNCCC12CN(Cc3ccccc3) | 378.550 | 0.57 |
| | | | CCOc(c4CCCC2)cccc4 | | |
| 3 | 6-(2,3-Dihydro-1,4-Benzodioxin-6-Yl)-3-[(4-Methoxyphenoxy) | 24824947 | n12c(nnc1COc(cc3)ccc3OC)sc(c | 396.430 | 0.60 |
| | Methyl][1,2,4]Triazolo[3,4-B][1,3,4]Thiadiazole | | (ccc(c45)OCCO4)c5)n2 | | |
| 4 | NH125 | 10436839 | CCCCCCCCCCCCCCCCn1c | 524.564 | 1.07 |
| | | | (C)[n+](Cc2ccccc2)cc1.[I-] | | |
| 5 | 6-(4-Me6-(4-Methylbenzyl)-2-Phenyl[1,2,4]Triazolo[1,5-C] | 20875688 | nl(nc(n2)c3ccccc3)c2c4c(cccc4) | 366.430 | 1.17 |
| | Quinazolin-5(6H)-One | | N(Cc(cc5)ccc5C)C1═O | | |
| 6 | Haloprogin | 3561 | C(COc(c1Cl)cc(c(Cl)c1)Cl)#CI | 361.395 | 1.44 |
| 7 | AC-93253 iodide, 2-[3-(1,3-Dihydro-1,3,3-trimethyl-2H-indol-2-ylidene)- | 16078948 | ccc2)/C3(C)C)[n+](CC)c(cccc4)c14.[I-] | 488.427 | 1.57 |
| | 1-propenyl]-3-ethyl-benzothiazolium iodide | | | | |
| 8 | C18H18ClN5O | 644215 | ClC1═CC(NC2═NC═CC(C3═CC═NC | 355.821 | 1.66 |
| | 3-[[4-[2-(3-chloroanilino)pyrimidin-4-yl]pyridin-2-yl]amino]propan-1-ol | | (NCCCO)═C3)═N2)═CC═C1 | | |
| 9 | N-(3-Chloro-4-Methylphenyl)-N'-(1-Methyl-5-Phenyl-1H-Imidazol-2-Yl)Urea | 22577915 | c1(NC(═O)Nc(ccc(c2Cl)C)c2) | 340.820 | 1.68 |
| | | | ncc(c3ccccc3)n1C | | |
| 10 | QNZ | 509554 | NC1═CC2═C(NCCC3═CC═C | 356.420 | 1.83 |
| | | | (OC4═CC═CC═C4)C═C3) | | |
| | | | N═CN═C2C═C1 | | |
| 11 | 1-benzyl-4-[(4-fluorophenyl)methylsulfanyl]-6,7-dihydro-5-cyclopenta[d]pyrimidin-2-one | 35343983 | N1═C(SC([H])([H])c(cc2)ccc2F) | 366.460 | 2.27 |
| | | | C3═C(N(Cc4ccccc4)C1═O)CCC3 | | |
| 12 | 1-(3,6-dibromocarbazol-9-yl)-3-(3-methylbutylamino)propan-2-ol | 2874418 | n1(CC(CNCCC(C)C)O)c2c(c(cc(Br) | 468.240 | 2.41 |
| | | | cc3)c13)cc(Br)cc2 | | |
| 13 | CD 1530 | 24868309 | OC(c1ccc(c2cc(c3cc2)cc(O)c | 398.494 | 2.46 |
| | | | (C45CC(CC6C4)CC(C5)C6)c3) | | |
| | | | cc1)═O | | |
| 14 | Tetrindole mesylate | 54792409 | CS(═O)(═O)O.C1CCC(c2cc(c3cc2) | 390.539 | 2.52 |
| | | | c(c(C4NCC5)n35)CCC4)CC1 | | |
| 15 | N-[3-(4-benzylpiperidin-1-yl)propyl]-9-methyl-4H-thieno[3,2-c]chromene-2-carboxamide, | 20873152 | c12c(COc(cccc3C)c13)cc(C(N | 460.640 | 2.53 |
| | MCULE-2046798638 | | CCCN4CCC(CC4)Cc5ccccc5)═O)s2 | | |
| 16 | NVP-TAE 226 | 9934347 | O═C(C1═C(C═CC═C1)NC2═NC | 468.936 | 2.57 |
| | | | (NC3═C(C═C(C═C3)N4C | | |
| | | | COCC4)OC)═NC═C2C1)NC | | |
| 17 | Calmidazolium chloride, R 24571 chloride | 644274 | [n+]1(C(c2ccc(cc2)Cl)c3ccc( | 687.698 | 2.63 |
| | | | Cl)cc3)ccn(CC(OCc4ccc(cc4 | | |
| | | | C1)Cl)c5ccc(cc5C1)C1)c1.[C1-] | | |
| 18 | 3,6-dimethyl-N-[(4-methylphenyl)methyl]-[1,2]oxazolo[5,4-d]pyrimidin-4-amine | 53004449 | c(o1)(nc(C)nc2NCc(cc3)ccc3C) | 268.320 | 2.73 |
| | | | c2c(n1)C | | |
| 19 | N-[2-(cyclohexen-1-yl)ethyl]-3,6-dimethyl-[1,2]oxazolo[5,4-d]pyrimidin-4-amine | 53004453 | c(o1)(nc(C)nc2NCCC3═CCCCC3) | 272.350 | 2.92 |
| | | | c2c(n1)C | | |
| 20 | 1-(3-chloro-2-methylphenyl)-1H-pyrrole-2,5-dione | 243573 | O═C1C═CC(═O)N1c1cccc(Cl)c1C | 221.640 | 2.95 |
| 21 | 1-benzyl-4-benzylsulfanyl-6,7-dihydro-5H-cyclopenta[d]pyrimidin-2-one | 35344693 | N1═C(SC([H])([H])c2ccccc2) | 348.470 | 3.00 |
| | | | C3═C(N(Cc4ccccc4)C1═O)CCC3 | | |
| 22 | CBR 5884 | 4674993 | O═C(C1═C(C)C(SC#N)═C(NC | 336.390 | 3.11 |
| | | | (C2═CC═CO2)═O)S1)OCC | | |
| 23 | N-[2-(cyclohexen-1-yl)ethyl]-3-ethyl-6-methyl-[1,2]oxazolo[5,4-d]pyrimidin-4-amine | 53004459 | c(o1)(nc(C)nc2NCCC3═CCCCC3) | 286.380 | 3.14 |
| | | | c2c(n1)CC | | |
| 24 | 6--(3-chloro-4-methylphenyl)-5--(2-fluorophenyl)-[1,2,5]oxadiazolo[3,4-b]pyrazine-5,6-diamine | 1097778 | c1(non2)c2nc(Nc(ccc(c3Cl)C)c3) | 370.780 | 3.15 |
| | | | c(Nc4ccccc4F)n1 | | |
| 25 | PluriSIn #1 (NSC 14613) | 225362 | c1nccc(C(NNc2ccccc2)═O)c1 | 213.235 | 3.1 |
| 26 | 1-(9-ethylcarbazol-3-yl)pyrrole-2,5-dione | 2731782 | CCn1c2ccc(cc2c2ccccc12) | 290.316 | 3.18 |
| | | | N1C(═O)C═CC1═O | | |
| 27 | 1-(4-Butyl-phenyl)-pyrrole-2,5-dione | 604282 | CCCCc1ccc(cc1)N1C(═O) | 229.274 | 3.22 |
| | | | C═CC1═O | | |
| 28 | Thonzonium bromide | 11102 | c(c1)(ccc(OC)c1)CN(CC[N+] | 591.725 | 3.39 |
| | | | (CCCCCCCCCCCCCCCC) | | |
| | | | (C)C)c2ncccn2.[Br-] | | |
| 29 | HSP990 (NVP-HSP990) | 16226182 | n1c(c2cc(F)ccc2[C@H]3Cc4c | 379.388 | 3.62 |
| | | | (c(C)nc(N)n4)C(═O)N3)cccc1OC | | |
| 30 | 2-[3-(3-methylbutoxy)phenoxy]-N-pyridin-4-ylacetamide | 2323706 | CC(C)CCOc1cccc(OCC(═O) | 314.379 | 3.94 |
| | | | Nc2ccncc2)c1 | | |
| 31 | N-[2-(cyclohexen-1-yl)ethyl]-3-ethyl-[1,2]oxazolo[5,4-d]pyrimidin-4-amine | 53004443 | c1(c2CC)c(ncnc1NCCC3═CC | \|272.350 | 4.09 |
| | | | CCC3)on2 | | |
| 32 | 1-(2-bromophenyl)-1H-pyrrole-2,5-dione | 709265 | O═C1C═CC(═O)N1c1ccccc1 | 252.064 | 4.61 |
| | | | Br | | |
| 33 | Adaptaquin | 3740671 | OC1═C(N═CC═C1)NC(C2═C | 377.820 | 4.63 |
| | | | (C3═C(C═C2)C═CC═N3)O) | | |
| | | | C4═CC═C(C═C4)C1 | | |
| 34 | JAK3 Inhibitor VI | 16760524 | c1ncc(c2cc(\C(═C\c3[nH]ccc3)\ | 383.421 | 4.67 |
| | | | C(═O)N4)c4cc2)cc1.CS(O) | | |
| | | | (═O)═O | | |
| 35 | Fingolimod | 107970 | CCCCCCCCC1═CC═C | 307.471 | 4.76 |
| | | | (CCC(CO)(N)CO)C═C1 | | |
| 36 | Cyto4A5 | 2809916 | COC(═O)c1sc(cc1N1C(═O) | 319.356 | 4.87 |
| | | | C═CC1═O)c1cccs1 | | |
| 37 | K-252a, Nocardiopsis sp. | 490561 | CC12C(CC(O1)N3C4═CC═C | 467.473 | 4.91 |
| | | | C═C4C5═C6C(═C7C8═CC═C | | |
| | | | C═C8N2C7═C53)CNC6═O) | | |
| | | | (c(═O)OC)O | | |
| 38 | N-[4-bromo-3-(trifluoromethyl)phenyl]thiophene-2-carboxamide | 2810425 | O═C(Nc1ccc(Br)c(c1)C(F)(F)F) | 350.154 | 4.96 |
| | | | c1cccs1 | | |
| 39 | BAY 11-7082 | 2301 | c1(ccc(C)cc1)S(═O)(═O)C═CC#N | 207.249 | 5.07 |
| 40 | Methiazole | 6604471 | c1(NC(OC)═O)[nH]c(c2n1)ccc | 265.336 | 5.08 |
| | | | (SC(C)C)c2 | | |
| 41 | 2-AAPA hydrate | None | CC(═O)N[C@@H](CSC(═S) | 518.650 | 5.14 |
| | | | NSc1ccc(cc1)SNC(═S)SC[C═O)O.O | | |
| 42 | 1-(3,5-Dimethylphenyl)-1H-pyrrole-2,5-dione | 243574 | Cc1cc(C)cc(c1)N1C(═O)C═CC1═O | 201.221 | 5.31 |
| 43 | MNS (3,4-Methylenedioxy-β-nitrostyrene, MDBN) | 94945 | c(OCO1)(cc(C═C[N+]([O-])═O)cc2)c12 | 193.156 | 5.31 |
| 44 | 1-methyl-N~6~-(2-methylphenyl)-N~4~-phenyl-1H-pyrazolo[3,4-d]pyrimidine-4,6-diamine, | 16456621 | c(n1C)(nc(Nc2ccccc2C) | 330.400 | 5.38 |
| | STK890868 | | nc3Nc4ccccc4)c3cn1 | | |
| 45 | 1-(4-butan-2-ylphenyl)pyrrole-2,5-dione | 2732151 | CCC(C)c1ccc(cc1)N1C(═O) | 229.274 | 5.49 |
| | | | C═CC1═O | | |
| 46 | Benzethonium chloride | 8478 | C(C)(C)(CC(C)(C)C)c1ccc | 448.094 | 5.52 |
| | | | (OCCOCC[N+](C)(C)Cc2ccccc2) | | |
| | | | cc1.[Cl-] | | |
| 47 | 6-(3,4-dimethoxyphenyl)-3-[(4-methoxyphenoxy)methyl]-[1,2,4]triazolo[3,4-b] | 44120785 | n12c(nnc1COc(cc3)ccc3OC) | 398.440 | 5.59 |
| | [1,3,4]thiadiazole | | sc(c4ccc(c(OC)c4)OC)n2 | | |
| 48 | Maleimide, N-phenethyl- | 243296 | O═C1C═CC(═O)N1CCc1ccccc1 | 201.221 | 5.63 |
| 49 | N-(2-phenylethyl)thieno[3,2-d]pyrimidin-4-amine | 2808903 | c1ccc(cc1)CCNc1ncnc2ccsc12 | 255.338 | 5.83 |
| 50 | 5-Nonyloxytryptamine | 23581825 | CCCCCCCCCOC1═CC2═C | 338.915 | 6.04 |
| | | | (C═C1)NC═C2CCN.Cl | | |
| 51 | 2-[[5-(2,4-dichlorophenyl)furan-2-yl]methylamino]-1-phenylethanol | 4724352 | c1(ccc(CN([H])CC(O)c2ccccc2)o1) | 398.720 | 6.09 |
| | | | c3ccc(cc3Cl)Cl | | |
| 52 | AS 1517499 | 10340781 | NC(═O)c1cnc(nc1NCc1ccccc1)NCCc1ccc(c(c1)Cl)O | 397.860 | 6.28 |
| 53 | 3-methyl-4-(3-phenylpyrrolidin-1-yl)-[1,2]oxazolo[5,4-d]pyrimidine | 20924730 | c1(ncnc(on2)c1c2C)N3CCC(c4ccccc4)C3 | 280.330 | 6.29 |
| 54 | BADGE | 2286 | CC(c1ccc(OCC20C2)cc1)(c3ccc | 340.413 | 6.42 |
| | | | (OCC40C4)cc3)C | | |
| 55 | 1-(1,3-benzodioxo1-5-yl)-N-[[5-(4-chlorophenyl)furan-2-yl]methyl]methanamine | 4723614 | c1(c2ccc(cc2)Cl)ccc(CN([H]) | 378.260 | 6.59 |
| | | | Cc(ccc(c34)OCO3)c4)o1 | | |
| 56 | Ethyl 1-(5-methylthieno[2,3-d]pyrimidin-4-yl)piperidine-4-carboxylate | 2809616 | CCOC(═O)C1CCN(CC1)c1nc | 305.395 | 6.60 |
| | | | nc2scc(C)c12 | | |
| 57 | Keratinocyte Differentiation Inducer | 11453158 | n1cc(ccn2c3cc(CCC#N)ccc3) | 429.471 | 6.84 |
| | | | c2nc1Nc4cc(OC)c(OC)c(OC)c4 | | |
| 58 | N-[(4-chlorophenyl)methyl]-1-(5,6-dimethylfuro[2,3-d]pyrimidin-4-yl) | 46289902 | c12c(ncnc1N(CC3)CCC3C(═O) | 398.900 | 6.87 |
| | piperidine-4-carboxamide | | NCc(cc4)ccc4Cl)oc(C)c2C | | |
| 59 | 1-(5-Chloro-2-methylphenyl)-4-[(1-{[2-(4-chlorophenyl)-5-methyl-1,3-oxazol-4-yl] | 16007906 | n1c(c2ccc(cc2)Cl)oc(C)c1CN | 527.500 | 6.93 |
| | methyl}piperidin-4-yl)carbonyl]piperazine | | (CC3)CCC3C(═O)N(CC4)CC | | |
| | | | N4c5c(C)ccc(Cl)c5 | | |
| 60 | 1-[4-[3-(trifluoromethyl)pyrazol-1-yl]phenyl]pyrrole-2,5-dione | 2809261 | O═C1C═CC(═O)N1c1ccc(cc1) | 307.227 | 7.06 |
| | | | n1ccc(n1)C(F)(F)F | | |
| 61 | 1-[4-(61-[4-(6-methyl-1,3-benzothiazol-2-yl)phenyl]-3-(4-methylpiperidin-1-yl) | 4115172 | N1(c(cc2)ccc2c3sc4c(ccc(C)c4) | 419.550 | 7.28 |
| | pyrrolidine-2,5-dione | | n3)C(═O)CC(N5CCC(CC5)C)C1═O | | |
| 62 | 1,3-dihydroxyxanthen-9-one | 5488193 | c12c(cc(O)cc1O)Oc(c3C2═O)cccc3 | 228.210 | 7.59 |
| 63 | N-(3-chN-(3-chloro-4-fluorophenyl)-4-[[2-(4-fluorophenyl) | 53006976 | n1c(ccnc1c2ccc(cc2)F)Nc(cc3) | 436.850 | 7.65 |
| | pyrimidin-4-yl]amino]benzamide | | ccc3C(═O)Nc(ccc(c4Cl)F)c4 | | |
| 64 | 6-chloro-12-[[4-[2-(dimethylamino)ethoxy]-3-methoxyphenyl]methyl]-2,19-dioxa-9,12- | 110075541 | c1(OCCN(C)C)ccc(CN(CCC | 582.130 | 7.75 |
| | diazatricyclo[18.4.0.03,8]tetracosa-1(24),3(8),4,6,20,22-hexaen-10-one | | CCCOc(c2O3)cccc2)CC(═O) | | |
| | | | Nc(c4)c3ccc4Cl)cc1OC | | |
| 65 | Tyrphostin 9 | 5614 | c1(C(C)(C)C)cc(C═C(C#N)C#N) | 282.380 | 7.87 |
| | | | cc(C(C)(C)C)c1O | | |
| 66 | 2-(6-chloro-2,4-dihydropyrido[3,2-h][1,3]benzoxazin-3-yl)-N,N-diethylethanamine | 2353946 | CCN(CC)CCN1COc2c(C1)cc | 319.829 | 8.08 |
| | | | (Cl)c1cccnc21 | | |
| 67 | 1-((4-Chlorophenyl)methyl)-1h-pyrrole-2,5-dione | 2392334 | Clc1ccc(cc1)CN1C(═O)C═CC1═O | 221.640 | 8.09 |
| 68 | 1-(1,3-benzodioxol-5-yl)-N-[[5-(4-bromophenyl)furan-2-yl]methyl]methanamine | 4723769 | c1(c2ccc(cc2)Br)ccc(CN([H]) | 422.710 | 8.10 |
| | | | Cc(ccc(c34)OCO3)c4)o1 | | |
| 69 | 2-(4-butoxyphenyl)-4-(4-ethylpiperazin-1-yl)pyrazolo[1,5-a]pyrazine | 50796547 | c12n(ccnc1N(CC3)CCN3CC) | 379.510 | 8.19 |
| | | | nc(c4ccc(cc4)OCCCC)c2 | | |
| 70 | 1-[3-(Methylsulfanyl)phenyl]-2,5-dihydro-1H-pyrrole-2,5-dione | 304757 | CSc1cccc(c1)N1C(═O)C═CC1═O | 219.260 | 8.20 |
| 71 | 1-(4-bromophenyl)-3-[2-(diethylamino)-4-methylquinolin-6-yl]urea | 46288508 | n1c(ccc(c2)NC(═O)Nc(cc3)cc | 427.350 | 8.38 |
| | | | c3Br)c2c(cc1N(CC)CC)C | | |
| 72 | N-[5-(2,4-dichlorophenyl)furan-2-yl]methyl]-1-pyridin-2-ylmethanamine | 4724345 | c1(ccc(CN([H])Cc2ccccn2)o1) | 369.680 | 8.46 |
| | | | c3ccc(cc3Cl)Cl | | |
| 73 | Lylamine hydrochloride | 71297107 | CC(C)C1═CC2═C(C═C1)C3 | 321.928 | 8.59 |
| | | | (CCCC(C3CC2)(C)CN)C.[C1-] | | |
| 74 | 4,6-Diphenyl-1-hydroxy-pyridine-2-one | 4370641 | On1c(═O)cc(cc1c1ccccc1)c1ccccc1 | 263.291 | 8.63 |
| 75 | 8-(4-fluorophenyl)-2-(4-methoxyphenyl)-N-(2-piperidin-1-ylethyl)quinoline-4-carboxamide | 20932539 | c12c(cccc1c3ccc(cc3)F)c | 483.590 | 8.74 |
| | MCULE-4343682937 | | (C(NCCN4CCCCC4)═O)cc | | |
| | | | (c5ccc(cc5)OC)n2 | | |
| 76 | 5-(4-methylphenyl)sulfanylquinazoline-2,4-diamine | 446245 | Cc1ccc(cc1)Sc1cccc2c1c(N)nc(n2)N | 282.360 | 8.75 |
| 77 | 2-chloro-4-(4-methylpiperidin-1-yl)quinazoline | 22692595 | c1(N2CCC(CC2)C)c3c(cccc3)nc(C1)n1 | 261.760 | 9.01 |
| 78 | 6-Phenoxybenzo[d]thiazol-2-amine | 2344996 | Nc1nc2ccc(Oc3ccccc3)cc2s1 | 242.296 | 9.20 |
| 79 | 3-[(Dimethylamino)methyl]-2,3-dihydrochromen-4-one;chloride | 45108735 | Cl.CN(C)CC1COc2ccccc2C1═O | 241.714 | 9.27 |
| 80 | 1-[2-(2,4-dichlorophenoxy)phenyl]pyrrole-2,5-dione | 2732171 | Clc1ccc(Oc2ccccc2N2C(═O) | 334.154 | 9.65 |
| | | | C═CC2═O)c(C1)c1 | | |
| 81 | Anisomycin | 31549 | CC(═O)OC1C(CNC1CC2═CC═C | 265.300 | 9.69 |
| | | | (C═C2)OC)O | | |
| 82 | 2-[[5-(2,5-dichlorophenyl)furan-2-yl]methylamino]-1-phenylethanol | 4970347 | c1(ccc(CN([H])CC(O)c2ccccc2)o1) | 398.720 | 9.78 |
| | | | c3cc(Cl)ccc3Cl | | |
| 83 | N-[5-tert-butyl-3-(4-fluorophenyl)-2-methylpyrazolo[1,5-a]pyrimidin-7-yl]- | 46299191 | c1(c(c2ccc(cc2)F)c(n3)C)n3c( | 369.490 | 9.92 |
| | N',N'-dimethylethane-1,2-diamine | | cc(C(C)(C)C)n1)NCCN(C)C | | |
| 84 | Staurosporine | 46864193 | CC12C(C(CC(O1)N3C4═CC═ | 466.500 | 9.92 |
| | | | CC═C4C5═C6C(═C7C8═CC═ | | |
| | | | CC═C8N2C7═C53)CNC6═O) | | |
| | | | NC)OC | | |
| 85 | N-[5-(4-bromophenyl)furan-2-yl]methyl]-1-(furan-2-yl)methanamine | 4723797 | c1(c2ccc(cc2)Br)ccc(CN([H]) | 368.660 | 9.96 |
| | | | Cc3ccco3)o1 | | |
| 86 | N-benzyl-3,6-dimethyl-[1,2]oxazolo[5,4-d]pyrimidin-4-amine | 53004444 | c(o1)(nc(C)nc2NCc3ccccc3)c | 254.290 | 9.99 |
| | | | 2c(n1)C | | |
| 87 | 4-benzyl-1″-(1-methylpiperidine-4-carbonyl)-2,3,4,5,7,8,9,10-octahydrospiro[1,4- | 110075516 | C(CC(C(N(CC1)CCC12CN | 503.719 | 10.07 |
| | benzoxazacyclododecine-6,4″-piperidine] | | (Cc3ccccc3)CCOc(c4CCCC2) | | |
| | | | cccc4)═O)C5)N(C)C5 | | |
| 88 | LE 135 | 10410894 | CN1c2c(cccc2)C(c3ccc | 438.561 | 10.08 |
| | | | (C(═O)O)cc3)═Nc4c1cc5c | | |
| | | | (C(C)(C)CCC5(C)C)c4 | | |
| 89 | AZ-960 | 56965903 | CC1═CC(═NN1)NC2═C | 354.357 | 10.36 |
| | | | (C═C(C(═N2)NC(C) | | |
| | | | C3═CC═C(C═C3)F)C#N)F | | |
| 90 | N,N-diphenyl-4-[(E)-1,2,4-triazol-4-yliminomethyl]aniline | 6903026 | c1ccc(cc1)N(c1ccc | 339.393 | 10.47 |
| | | | (\C═N/n2cnnc2)cc1) | | |
| | | | c1ccccc1 | | |
| 91 | N-{[2-(4-ethylphenyl)imidazo[1,2-a]pyridin-3-yl]methyl}-N-(3-methylbenzyl)amine, | 20956381 | n(c(cccc1)n1c2CNC | 355.490 | 10.65 |
| | MCULE-1645297855 | | c3cccc(C)c3)c2c4ccc | | |
| | | | (cc4)CC | | |

### Example 2

The following is a non-limiting example of the present invention. It is to be understood that said example is not intended to limit the present invention in any way. Equivalents or substitutes are within the scope of the present invention.

### Example 2.1 Oral Administration of a Composition Comprising an Antifungal Agent, as Described Herein, to Treat Cryptococcus

A 60-year-old man visits his physician, troubled by persistent breathlessness and a nagging cough. Recognizing the situation's urgency, the doctor orders a comprehensive blood analysis to uncover the root cause of his symptoms. Upon receiving the test results, it is revealed that the man has a Cryptococcus infection. The physician wastes no time and prescribes an oral composition comprising an antifungal agent, as described herein, designed to treat Cryptococcus. The man adheres to the prescribed regimen and experiences a gradual alleviation of his symptoms with each dose. No side effects reported.

### Example 2.2 Oral Administration of a Composition Comprising an Antifungal Agent, as Described Herein, to Treat Cryptococcus Meningitis

A 75-year-old man seeks medical attention, troubled by persistent headaches, nausea, and a lingering cough. Sensing the urgency of his condition, the physician promptly orders a comprehensive blood analysis to ascertain the underlying cause of his symptoms. The man's test results reveal that he has Cryptococcus meningitis. The physician prescribes an oral composition comprising an antifungal agent, as described herein, designed to treat fungal infections caused by Cryptococcus. The man adheres to the prescribed regimen and experiences a gradual alleviation of his symptoms with each dose. No side effects reported.

### Example 2.3 Inhaled Administration of a Composition Comprising an Antifungal Agent, as Described Herein, to Treat Cryptococcus

A 45-year-old woman, troubled by shortness of breath, a nagging cough, and an accompanying fever, seeks medical attention. Because the patient is immunocompromised, the doctor quickly orders a comprehensive blood analysis to uncover the root cause of her symptoms. Upon receiving the test results, it is revealed that she has a Cryptococcus infection. Recognizing the gravity of the situation, the physician wastes no time and devises a targeted treatment plan. Instead of opting for oral medication, the physician prescribes an inhaled composition comprising an antifungal agent, as described herein, specifically designed to treat Cryptococcus. The woman adheres to the prescribed regimen, administering the therapeutic composition via inhalation. With each dose, she experiences a gradual alleviation of her symptoms. No side effects reported.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1.-24. (canceled)

25. A composition for treating and/or preventing a fungal infection caused by a fungus in the Cryptococcus genus, the composition comprising an antifungal agent in a pharmaceutically acceptable carrier.

26. The composition of claim 25, wherein the fungal infection is caused by Cryptococcus neoformans.

27. The composition of claim 25, wherein the fungal infection is caused by Cryptococcus gattii.

28. The composition of claim 25, wherein the fungal infection is cryptococcosis.

29. The composition of claim 25, wherein the fungal infection is cryptococcal meningitis.

30. The composition of claim 25, wherein the antifungal agent is selected from a group consisting of: -[2-(4-chlorophenyl)ethyl]thieno[2,3-d]pyrimidin-4-amine, 4-benzyl-2,3,4,5,7,8,9,10-octahydrospiro[1,4-benzoxazacyclododecine-6,4″-piperidine], 6-(2,3-Dihydro-1,4-Benzodioxin-6-Yl)-3-[(4-Methoxyphenoxy)Methyl][1,2,4]Triazolo[3,4-B][1,3,4]Thiadiazole, NH125, 6-(4-Me6-(4-Methylbenzyl)-2-Phenyl[1,2,4]Triazolo[1,5-C]Quinazolin-5(6H)-One, haloprogin, AC-93253 iodide (2-[3-(1,3-Dihydro-1,3,3-trimethyl-2H-indol-2-ylidene)-1-propenyl]-3-ethyl-benzothiazolium iodide), C18H18ClN5O, 3-[[4-[2-(3-chloroanilino)pyrimidin-4-yl]pyridin-2-yl]amino]propan-1-ol, N-(3-Chloro-4-Methylphenyl)-N′-(1-Methyl-5-Phenyl-1H-Imidazol-2-Yl)Urea, QNZ, CD 1530, Tetrindole mesylate, 1-benzyl-4-[(4-fluorophenyl)methylsulfanyl]-6,7-dihydro-5-cyclopenta[d]pyrimidin-2-one, 1-(3,6-dibromocarbazol-9-yl)-3-(3-methylbutylamino)propan-2-ol, N-[3-(4-benzylpiperidin-1-yl)propyl]-9-methyl-4H-thieno[3,2-c]chromene-2-carboxamide, MCULE-2046798638, NVP-TAE 226, Calmidazolium chloride, R 24571 chloride, 3,6-dimethyl-N-[(4-methylphenyl)methyl]-[1,2]oxazolo [5,4-d]pyrimidin-4-amine, N-[2-(cyclohexen-1-yl)ethyl]-3,6-dimethyl-[1,2]oxazolo [5,4-d]pyrimidin-4-amine, 1-(3-chloro-2-methylphenyl)-1H-pyrrole-2,5-dione, 1-benzyl-4-benzylsulfanyl-6,7-dihydro-5H-cyclopenta[d]pyrimidin-2-one, CBR 5884, N-[2-(cyclohexen-1-yl)ethyl]-3-ethyl-6-methyl-[1,2]oxazolo[5,4-d]pyrimidin-4-amine, 6-(3-chloro-4-methylphenyl)-5-(2-fluorophenyl)-[1,2,5]oxadiazolo[3,4-b]pyrazine-5,6-diamine, PluriSIn #1 (NSC 14613), 1-(9-ethylcarbazol-3-yl)pyrrole-2,5-dione, 1-(4-Butyl-phenyl)-pyrrole-2,5-dione, Thonzonium bromide, HSP990 (NVP-HSP990), 2-[3-(3-methylbutoxy)phenoxy]-N-pyridin-4-ylacetamide, N-[2-(cyclohexen-1-yl)ethyl]-3-ethyl-[1,2]oxazolo[5,4-d]pyrimidin-4-amine, 1-(2-bromophenyl)-1H-pyrrole-2,5-dione, Adaptaquin, JAK3 Inhibitor VI, Fingolimod, Cyto4A5, K-252a (Nocardiopsis sp.), N-[4-bromo-3-(trifluoromethyl)phenyl]thiophene-2-carboxamide, BAY 11-7082, Methiazole, 2-AAPA hydrate, 1-(3,5-Dimethylphenyl)-1H-pyrrole-2,5-dione, MNS (3,4-Methylenedioxy-p-nitrostyrene, MDBN), 1-methyl-N-6-(2-methylphenyl)-N~4~-phenyl-1H-pyrazolo[3,4-d]pyrimidine-4,6-diamine, STK890868, 1-(4-butan-2-ylphenyl) pyrrole-2,5-dione, Benzethonium chloride, 6-(3,4-dimethoxyphenyl)-3-[(4-methoxyphenoxy)methyl]-[1,2,4]triazolo[3,4-b][1,3,4]thiadiazole, Maleimide (N-phenethyl-N-(2-phenylethyl)thieno[3,2-d]pyrimidin-4-amine), 5-Nonyloxytryptamine, 2-[[5-(2,4-dichlorophenyl)furan-2-yl]methylamino]-1-phenylethanol, AS 1517499, 3-methyl-4-(3-phenylpyrrolidin-1-yl)-[1,2]oxazolo[5,4-d]pyrimidine, BADGE, 1-(1,3-benzodioxol-5-yl)-N-[[5-(4-chlorophenyl)furan-2-yl]methyl]methanamine, Ethyl 1-(5-methylthieno[2,3-d]pyrimidin-4-yl)piperidine-4-carboxylate, Keratinocyte Differentiation Inducer, N-[(4-chlorophenyl)methyl]-1-(5,6-dimethylfuro[2,3-d]pyrimidin-4-yl)piperidine-4-carboxamide, 1-(5-Chloro-2-methylphenyl)-4-[(1-{[2-(4-chlorophenyl)-5-methyl-1,3-oxazol-4-yl]methyl}piperidin-4-yl)carbonyl]piperazine, 1-[4-[3-(trifluoromethyl)pyrazol-1-yl]phenyl]pyrrole-2,5-dione, 1-[4-(61-[4-(6-methyl-1,3-benzothiazol-2-yl)phenyl]-3-(4-methylpiperidin-1-yl)pyrrolidine-2,5-dion, 1,3-dihydroxyxanthen-9-one, N-(3-chN-(3-chloro-4-fluorophenyl)-4-[[2-(4-fluorophenyl) pyrimidin-4-yl]amino]benzamide, 6-chloro-12-[[4-[2-(dimethylamino)ethoxy]-3-methoxyphenyl]methyl]-2,19-dioxa-9,12-diazatricyclo[18.4.0.03,8]tetracosa-1(24),3(8),4,6,20,22-hexaen-10-one, Tyrphostin 9, 2-(6-chloro-2,4-dihydropyrido[3,2-h][1,3]benzoxazin-3-yl)-N,N-diethylethanamine, 1-((4-Chlorophenyl)methyl)-1h-pyrrole-2,5-dione, 1-(1,3-benzodioxol-5-yl)-N-[[5-(4-bromophenyl)furan-2-yl]methyl]methanamine, 2-(4-butoxyphenyl)-4-(4-ethylpiperazin-1-yl)pyrazolo[1,5-a]pyrazine, 1-[3-(Methylsulfanyl)phenyl]-2,5-dihydro-1H-pyrrole-2,5-dione, 1-(4-bromophenyl)-3-[2-(diethylamino)-4-methylquinolin-6-yl]urea, N-[[5-(2,4-dichlorophenyl)furan-2-yl]methyl]-1-pyridin-2-ylmethanamine, Lylamine hydrochloride, 4,6-Diphenyl-1-hydroxy-pyridine-2-one, 8-(4-fluorophenyl)-2-(4-methoxyphenyl)-N-(2-piperidin-1-ylethyl) quinoline-4-carboxamide MCULE-4343682937, 5-(4-methylphenyl)sulfanylquinazoline-2,4-diamine, 2-chloro-4-(4-methylpiperidin-1-yl)quinazoline, 6-Phenoxybenzo[d]thiazol-2-amine, 3-[(Dimethylamino)methyl]-2,3-dihydrochromen-4-one;chloride, 1-[2-(2,4-dichlorophenoxy)phenyl]pyrrole-2,5-dione, Anisomycin, 2-[[5-(2,5-dichlorophenyl)furan-2-yl]methylamino]-1-phenylethanol, N-[5-tert-butyl-3-(4-fluorophenyl)-2-methylpyrazolo[1,5-a]pyrimidin-7-yl]-N′,N′-dimethylethane-1,2-diamine, Staurosporine, N-[[5-(4-bromophenyl)furan-2-yl]methyl]-1-(furan-2-yl)methanamine, N-benzyl-3,6-dimethyl-[1,2]oxazolo[5,4-d]pyrimidin-4-amine, 4-benzyl-1″-(1-methylpiperidine-4-carbonyl)-2,3,4,5,7,8,9,10-octahydrospiro[1,4-benzoxazacyclododecine-6,4″-piperidine], LE 135, AZ-960, N,N-diphenyl-4-[(E)-1,2,4-triazol-4-yliminomethyl]aniline, and N-{[2-(4-ethylphenyl)imidazo[1,2-a]pyridin-3-yl]methyl}-N-(3-methylbenzyl)amine (MCULE-1645297855).

31. The composition of claim 30, wherein the antifungal agent has an IC₅₀ less than 10 uM.

32. A method for preventing and/or treating a fungal infection caused by a fungus in the Cryptococcus genus in a subject in need thereof, the method comprising administering a therapeutically effective amount of an antifungal composition to the subject, wherein the antifungal composition comprises an antifungal agent in a pharmaceutically acceptable carrier.

33. The method of claim 32, wherein the fungal infection is caused by Cryptococcus neoformans.

34. The method of claim 32, wherein the fungal infection is caused by Cryptococcus gattii.

35. The method of claim 32, wherein the fungal infection is cryptococcosis.

36. The method of claim 32, wherein the fungal infection is cryptococcal meningitis.

37. The method of claim 32, wherein the antifungal agent is selected from a group consisting of: N-[2-(4-chlorophenyl)ethyl]thieno[2,3-d]pyrimidin-4-amine, 4-benzyl-2,3,4,5,7,8,9,10-octahydrospiro[1,4-benzoxazacyclododecine-6,4″-piperidine],
6-(2,3-Dihydro-1,4-Benzodioxin-6-Yl)-3-[(4-Methoxyphenoxy)Methyl][1,2,4]Triazolo[3,4-B][1,3,4]Thiadiazole, NH125, 6-(4-Me6-(4-Methylbenzyl)-2-Phenyl[1,2,4]Triazolo [1,5-C]Quinazolin-5(6H)-One, haloprogin, AC-93253 iodide (2-[3-(1,3-Dihydro-1,3,3-trimethyl-2H-indol-2-ylidene)-1-propenyl]-3-ethyl-benzothiazolium iodide), C18H18ClN5O, 3-[[4-[2-(3-chloroanilino)pyrimidin-4-yl]pyridin-2-yl]amino]propan-1-ol, N-(3-Chloro-4-Methylphenyl)-N′-(1-Methyl-5-Phenyl-1H-Imidazol-2-Yl)Urea, QNZ, 1-benzyl-4-[(4-fluorophenyl)methylsulfanyl]-6,7-dihydro-5-cyclopenta[d]pyrimidin-2-one, 1-(3,6-dibromocarbazol-9-yl)-3-(3-methylbutylamino)propan-2-ol, CD 1530, Tetrindole mesylate, N-[3-(4-benzylpiperidin-1-yl)propyl]-9-methyl-4H-thieno [3,2-c]chromene-2-carboxamide, MCULE-2046798638, NVP-TAE 226, Calmidazolium chloride, R 24571 chloride, 3,6-dimethyl-N-[(4-methylphenyl)methyl]-[1,2]oxazolo [5,4-d]pyrimidin-4-amine, N-[2-(cyclohexen-1-yl)ethyl]-3,6-dimethyl-[1,2]oxazolo [5,4-d]pyrimidin-4-amine, 1-(3-chloro-2-methylphenyl)-1H-pyrrole-2,5-dione, 1-benzyl-4-benzylsulfanyl-6,7-dihydro-5H-cyclopenta[d]pyrimidin-2-one, CBR 5884, N-[2-(cyclohexen-1-yl)ethyl]-3-ethyl-6-methyl-[1,2]oxazolo[5,4-d]pyrimidin-4-amine, 6-(3-chloro-4-methylphenyl)-5-(2-fluorophenyl)-[1,2,5]oxadiazolo[3,4-b]pyrazine-5,6-diamine, PluriSIn #1 (NSC 14613), 1-(9-ethylcarbazol-3-yl)pyrrole-2,5-dione, 1-(4-Butyl-phenyl)-pyrrole-2,5-dione, Thonzonium bromide, HSP990 (NVP-HSP990), 2-[3-(3-methylbutoxy)phenoxy]-N-pyridin-4-ylacetamide, N-[2-(cyclohexen-1-yl)ethyl]-3-ethyl-[1,2]oxazolo[5,4-d]pyrimidin-4-amine, 1-(2-bromophenyl)-1H-pyrrole-2,5-dione, Adaptaquin, JAK3 Inhibitor VI, Fingolimod, Cyto4A5, K-252a (Nocardiopsis sp.), N-[4-bromo-3-(trifluoromethyl)phenyl]thiophene-2-carboxamide, BAY 11-7082, Methiazole, 2-AAPA hydrate, 1-(3,5-Dimethylphenyl)-1H-pyrrole-2,5-dione, MNS (3,4-Methylenedioxy-β-nitrostyrene, MDBN), 1-methyl-N~6~-(2-methylphenyl)-N~4~-phenyl-1H-pyrazolo[3,4-d]pyrimidine-4,6-diamine, STK890868, 1-(4-butan-2-ylphenyl) pyrrole-2,5-dione, Benzethonium chloride, 6-(3,4-dimethoxyphenyl)-3-[(4-methoxyphenoxy)methyl]-[1,2,4]triazolo[3,4-b][1,3,4]thiadiazole, Maleimide (N-phenethyl-N-(2-phenylethyl)thieno[3,2-d]pyrimidin-4-amine), 5-Nonyloxytryptamine, 2-[[5-(2,4-dichlorophenyl)furan-2-yl]methylamino]-1-phenylethanol, AS 1517499, 3-methyl-4-(3-phenylpyrrolidin-1-yl)-[1,2]oxazolo[5,4-d]pyrimidine, BADGE, 1-(1,3-benzodioxol-5-yl)-N-[[5-(4-chlorophenyl)furan-2-yl]methyl]methanamine, Ethyl 1-(5-methylthieno[2,3-d]pyrimidin-4-yl)piperidine-4-carboxylate, Keratinocyte Differentiation Inducer, N-[(4-chlorophenyl)methyl]-1-(5,6-dimethylfuro[2,3-d]pyrimidin-4-yl)piperidine-4-carboxamide, 1-(5-Chloro-2-methylphenyl)-4-[(1-{[2-(4-chlorophenyl)-5-methyl-1,3-oxazol-4-yl]methyl}piperidin-4-yl)carbonyl]piperazine, 1-[4-[3-(trifluoromethyl)pyrazol-1-yl]phenyl]pyrrole-2,5-dione, 1-[4-(61-[4-(6-methyl-1,3-benzothiazol-2-yl)phenyl]-3-(4-methylpiperidin-1-yl)pyrrolidine-2,5-dion, 1,3-dihydroxyxanthen-9-one, N-(3-chN-(3-chloro-4-fluorophenyl)-4-[[2-(4-fluorophenyl) pyrimidin-4-yl]amino]benzamide, 6-chloro-12-[[4-[2-(dimethylamino)ethoxy]-3-methoxyphenyl]methyl]-2,19-dioxa-9,12-diazatricyclo[18.4.0.03,8]tetracosa-1(24),3(8),4,6,20,22-hexaen-10-one, Tyrphostin 9, 2-(6-chloro-2,4-dihydropyrido[3,2-h][1,3]benzoxazin-3-yl)-N,N-diethylethanamine, 1-((4-Chlorophenyl)methyl)-1h-pyrrole-2,5-dione, 1-(1,3-benzodioxol-5-yl)-N-[[5-(4-bromophenyl)furan-2-yl]methyl]methanamine, 2-(4-butoxyphenyl)-4-(4-ethylpiperazin-1-yl)pyrazolo[1,5-a]pyrazine, 1-[3-(Methylsulfanyl)phenyl]-2,5-dihydro-1H-pyrrole-2,5-dione, 1-(4-bromophenyl)-3-[2-(diethylamino)-4-methylquinolin-6-yl]urea, N-[[5-(2,4-dichlorophenyl)furan-2-yl]methyl]-1-pyridin-2-ylmethanamine, Lylamine hydrochloride, 4,6-Diphenyl-1-hydroxy-pyridine-2-one, 8-(4-fluorophenyl)-2-(4-methoxyphenyl)-N-(2-piperidin-1-ylethyl) quinoline-4-carboxamide MCULE-4343682937, 5-(4-methylphenyl)sulfanylquinazoline-2,4-diamine, 2-chloro-4-(4-methylpiperidin-1-yl)quinazoline, 6-Phenoxybenzo[d]thiazol-2-amine, 3-[(Dimethylamino)methyl]-2,3-dihydrochromen-4-one;chloride, Anisomycin, 1-[2-(2,4-dichlorophenoxy)phenyl]pyrrole-2,5-dione, 2-[[5-(2,5-dichlorophenyl) furan-2-yl]methylamino]-1-phenylethanol, Staurosporine, N-[5-tert-butyl-3-(4-fluorophenyl)-2-methylpyrazolo[1,5-a]pyrimidin-7-yl]-N′,N′-dimethylethane-1,2-diamine, N-[[5-(4-bromophenyl)furan-2-yl]methyl]-1-(furan-2-yl)methanamine, N-benzyl-3,6-dimethyl-[1,2]oxazolo[5,4-d]pyrimidin-4-amine, 4-benzyl-1″-(1-methylpiperidine-4-carbonyl)-2,3,4,5,7,8,9,10-octahydrospiro[1,4-benzoxazacyclododecine-6,4″-piperidine], LE 135, AZ-960, N,N-diphenyl-4-[(E)-1,2,4-triazol-4-yliminomethyl]aniline, and N-{[2-(4-ethylphenyl)imidazo[1,2-a]pyridin-3-yl]methyl}-N-(3-methylbenzyl)amine (MCULE-1645297855).

38. The method of claim 37, wherein the antifungal agent has an IC₅₀ less than 10 uM.
